(19) United States
(12) Patent Application Publication
(10) Pub. No.: US 2018/0355298 A1
(43) Pub. Date: Dec. 13, 2018

(54) MULTI-ORGAN CELL CULTURE SYSTEM AND METHODS OF USE THEREOF

(71) Applicant: The Regents of the University of California, Oakland, CA (US)

(72) Inventors: Peter Loskill, Berkeley, CA (US); Anurag Mathur, Berkeley, CA (US); Kevin E. Healy, Moraga, CA (US); Luke P. Lee, Orinda, CA (US)

(21) Appl. No.: 15/536,086

(22) PCT Filed: Dec. 14, 2015
(86) PCT No.: PCT/US15/65607

§ 371 Date: Jun. 14, 2017

Related U.S. Application Data
(60) Provisional application No. 62/091,840, filed on Dec. 15, 2014.

(51) Int. Cl. C12M 3/00; B01L 3/00; C12M 3/06; C12M 1/00; C12M 1/36; C12M 1/34; C12N 5/074; G01N 33/50

(52) U.S. Cl. CPC C12M 21/08; B01L 3/502715; C12M 23/16; C12M 23/58; B01L 2200/027; C12M 41/44; C12N 5/0607; G01N 33/5073; C12M 41/48

(57) ABSTRACT

Multi-organ cell culture systems and methods are provided. Aspects of the cell culture systems include at least two microfluidic cell culture units configured to culture a plurality of cells, one or more connectors configured to fluidly connect the microfluidic cell culture units to one another, a cell culture medium configured to support the growth of a plurality of different cell types, and a controller configured to move the cell culture medium at a specified volumetric flow rate between the microfluidic cell culture units. The subject systems and methods find use in a variety of applications, including in vitro evaluation of candidate agents for toxicity and efficacy, in vitro models of disease, and in vitro models for fundamental studies of biological systems.

### CROSS-REFERENCE

This application claims the benefit of U.S. Provisional Patent Application No. 62/091,840, filed Dec. 15, 2014, which application is incorporated herein by reference in its entirety.

### STATEMENT REGARDING FEDERALLY SPONSORED RESEARCH

This invention was made with Government support under Grant Number TR000487 awarded by the National Institutes of Health. The Government has certain rights in the invention.

### INTRODUCTION

There is an urgent need in the pharmaceutical industry to effectively and efficiently screen potential drug compounds during early stages of investigation to assess both effectiveness and toxicity. With the discovery of patient-specific human induced pluripotent stem (iPS) cells, it is now possible to develop in vitro disease-specific model tissues and organs to be used for high content drug screening and patient-specific medicine. By mimicking the dimensions and cellular arrangement of minimal functional units of human organs, cell culture units consisting of model tissue incorporated into microfluidic systems have been developed for various organ types. These model tissues have been generated by either differentiating pluripotent stem cells inside a cell culture system by directly introducing pre-differentiated organ-specific cells into a cell culture system, or by adding differentiated tissue-specific cells from human donors. For drug-screening and further pharmaceutical applications, it is inevitable that multiple organs will need to be connected to form a multi-organ system fed by a common medium. Organ-organ interactions, side effects, and metabolite toxicity can then be detected and studied. The present disclosure meets these and other needs.

### SUMMARY

Multi-organ cell culture systems and methods are provided. Aspects of the cell culture systems include at least two microfluidic cell culture units configured to culture a plurality of cells, one or more connectors configured to fluidly connect the microfluidic cell culture units to one another, a cell culture medium configured to support the growth of a plurality of different cell types, and a controller configured to move the cell culture medium at a specified volumetric flow rate between the microfluidic cell culture units. The subject systems and methods find use in a variety of applications, including in vitro evaluation of candidate agents for toxicity and efficacy, in vitro models of disease, and in vitro models for fundamental studies of biological systems.

### BRIEF DESCRIPTION OF THE DRAWINGS

FIG. 1 shows a master chip that includes a plurality of individual microfluidic cell culture units.

FIGS. 2A-C show example embodiments of connectors.

FIGS. 3A and 3B show schematic depictions of different phases of a method involving the subject devices and systems.

FIGS. 4A and 4B show schematic depictions of the requirements of the subject devices and systems.

FIG. 5 is a schematic depiction of components of the subject devices and systems.

FIGS. 6A-E depict the characterization of components of the subject devices and systems.

FIGS. 7A-C are schematic depictions of the general procedure and depict an outcome of using subject devices and systems.

### DEFINITIONS

The term “induced pluripotent stem cell” (or “iPS cell”), as used herein, refers to a stem cell induced from a somatic cell, e.g., a differentiated somatic cell, and that has a higher potency than said somatic cell. iPS cells are capable of self-renewal and differentiation into mature cells, e.g., cells of mesodermal lineage or cardiomyocytes. iPS cells may also be capable of differentiation into cardiac progenitor cells.

As used herein, the term “stem cell” refers to an undifferentiated cell that that is capable of self-renewal and differentiation into one or more mature cells, e.g., cells of a mesodermal lineage, cardiomyocytes, or progenitor cells. The stem cell is capable of self-maintenance, meaning that with each cell division, one daughter cell will also be a stem cell. Stem cells can be obtained from embryonic, fetal, post-natal, juvenile or adult tissue. The term “progenitor cell”, as used herein, refers to an undifferentiated cell derived from a stem cell, and is not itself a stem cell. Some progenitor cells can produce progeny that are capable of differentiating into more than one cell type.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is a human. In some embodiments, the individual is a murine.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an iPS cell” includes a plurality of such cells and reference to “the microfluidic cell culture unit” includes reference to one or more microfluidic cell culture units and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Multi-organ cell culture systems and methods are provided. Aspects of the cell culture systems include at least two microfluidic cell culture units configured to culture a plurality of cells, one or more connectors configured to fluidly connect the microfluidic cell culture units to one another, a cell culture medium configured to support the growth of a plurality of different cell types, and a controller configured to move the cell culture medium at a specified volumetric flow rate between the microfluidic cell culture units. The subject systems and methods find use in a variety of applications, including in vitro evaluation of candidate agents for toxicity and efficacy, in vitro models of disease, and in vitro models for fundamental studies of biological systems.

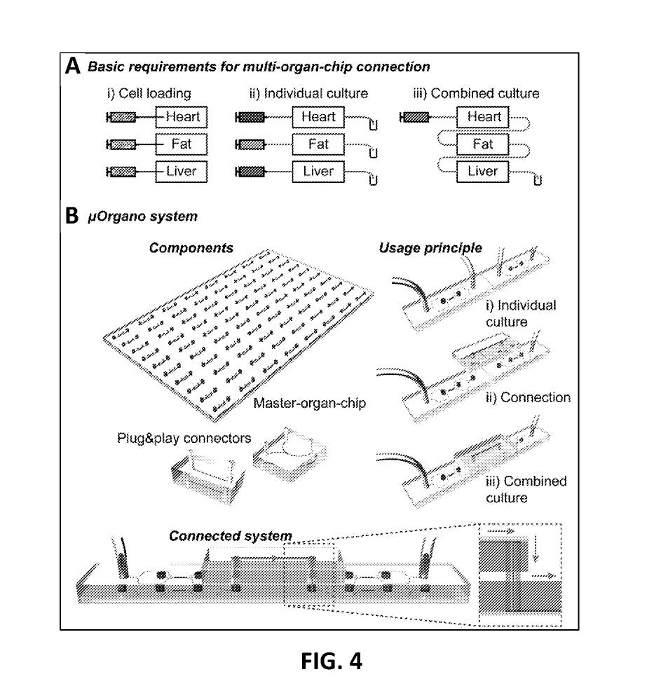

The present disclosure provides a multi-organ cell culture system. A multi-organ cell culture system of the present disclosure is also referred to as a “multi-organ μOrgano system,” or simply “μOrgano system.” The μOrgano system is a microphysiological system (MPS). MPS are also referred to in the art as “organ-on-a-chip” systems. The present multi-organ μOrgano system is customizable, and enables fluidic control of microliter (μL) volumes. The present multi-organ μOrgano system is specifically designed to connect multiple organ-on-a-chip (μ-organs) systems into multi-organ-chips. The present μOrgano system is a plug & play system that allows for: i) separate loading of different cell types; ii) temporal control of individual culture of cells for differentiation and tissue development; and, iii) subsequent temporal control of fluidic connections of the individual tissues, as depicted in FIG. 4A-4B.

### Microfluidic Cell Culture Units

Aspects of the disclosure include microfluidic cell culture units that are adapted for receiving and culturing a plurality of cells therein. Microfluidic cell culture units in accordance with embodiments of the invention are three-dimensional structures that are configured to provide an environment that is suitable for culturing cells. The subject microfluidic cell culture units are also configured to deliver a cell culture medium to the cells that are cultured within the units.

Microfluidic cell culture units in accordance with embodiments of the invention include one or more cell culture chambers. A cell culture chamber may have any of a variety of geometries and/or dimensions that are suitable for receiving and culturing cells therein, and include a base and one or more walls that define the boundaries of the chamber. In some embodiments, a cell culture chamber may have a circular, oval, square, rectangular or hexagonal geometry. In certain embodiments, a microfluidic cell culture unit may include from 1 to 10 individual cell culture chambers, such as 2, 3, 4, 5, 6, 7, 8 or 9 individual cell culture chambers. In some embodiments, two or more individual cell culture chambers may be fluidly connected to one another in series and/or in parallel.

The distance from the base of the cell culture chamber to the top of the walls defines the height of the chamber. In some embodiments, the height of the chamber ranges from 30 to 200 μm, such as 40, 50, 60, 70, 80, 90, 100, 110, 120, 130, 140, 150, 160, 170, 180 or 190 μm; e.g., the height of the chamber can range from about 30 μm to about 50 μm, from about 50 μm to about 75 μm, from about 75 μm to about 100 μm, from about 100 μm to about 125 μm, from about 125 μm to about 150 μm, from about 150 μm to about 175 μm, or from about 175 μm to about 200 μm.

In some embodiments, a cell culture chamber includes a channel that extends from one end of the cell culture chamber to another end of the cell culture chamber. In some embodiments, a cell culture channel is a three-dimensional structure that includes a base and two walls that extend from a first end to a second end of the chamber. The first end of the cell culture chamber is referred to as the “inlet end” and the second end of the cell culture chamber is referred to as the “outlet end.” The distance from the inlet end to the outlet end defines the length of the cell culture chamber.

In some embodiments, the length of the cell culture chamber ranges from 0.2 mm to 5 mm, such as 0.4, 0.6, 0.8, 1, 1.5, 2, 2.5, 3, 3.5, 4, or 4.5 mm; e.g., the length of the cell culture chamber can range from 0.2 mm to about 1 mm, from about 1 mm to about 2 mm, from about 2 mm to about 3 mm, from about 3 mm to about 4 mm, or from about 4 mm to about 5 mm

The distance between the two walls in the direction that is perpendicular to the length of the channel defines the width of the channel. In some embodiments, the width of the cell culture channel ranges from 30 μm to 200 μm, such as 40, 50, 60, 70, 80, 90, 100, 110, 120, 130, 140, 150, 160, 170, 180 or 190 μm; e.g., the width of the cell culture channel can range from about 30 μm to about 50 μm, from about 50 μm to about 75 μm, from about 75 μm to about 100 μm, from about 100 μm to about 125 μm, from about 125 μm to about 150 μm, from about 150 μm to about 175 μm, or from about 175 μm to about 200 μm. The distance from the base of the channel to the top of the walls defines the height of the cell loading channel. In some embodiments, the height of the channel ranges from 30 to 200 μm, such as 40, 50, 60, 70, 80, 90, 100, 110, 120, 130, 140, 150, 160, 170, 180 or 190 μm; e.g., the height of the cell culture channel can range from about 30 μm to about 50 μm, from about 50 μm to about 75 μm, from about 75 μm to about 100 μm, from about 100 μm to about 125 μm, from about 125 μm to about 150 μm, from about 150 μm to about 175 μm, or from about 175 μm to about 200 μm.

In some embodiments, a cell culture chamber may have a circular geometry with a radius that ranges from 100 to 500 μm, such as 150, 200, 250, 300, 350, 400, or 450 μm.

Microfluidic cell culture units in accordance with embodiments of the invention include at least one media channel that is configured to contain and transport a cell culture medium therein. The media channels are three-dimensional structures and may have any of a variety of geometries and dimensions that are suitable for transporting a cell culture medium. In some embodiments, a microfluidic cell culture unit includes two media channels, each disposed along one side of a cell culture channel. In certain embodiments, a microfluidic cell culture device may include a membrane that separates a cell culture chamber from one or more media channels, and which is configured to allow diffusion of one or more media components through the membrane to reach a plurality of cells that are cultured within the cell culture chamber.

Membranes in accordance with embodiments of the invention may have any suitable pore size and distribution, and may be configured to restrict cells from passing through the membrane while freely allowing one or more cell culture media components to pass through the membrane pores. In some embodiments, a membrane may have a pore size that ranges from 0.2 to 5 μm, such as 0.5, 1, 1.5, 2, 2.5, 3, 3.5, 4, or 4.5 μm. Membranes in accordance with embodiments of the invention may include any suitable materials, including but not limited to: polycarbonate (PC); polyester (e.g., polyethylene terephthalate (PET); polytetrafluoroethylene (PTFE); and the like.

In some embodiments, a media channel includes a base and two walls that extend from a first end of a channel to a second end of a channel. The distance from the first end of the media channel to the second end of the media channel defines the length of the media channel. In some embodiments, the length of each media channel is greater than or equal to the length of the cell culture channel. The distance between the two walls of the media channel in the direction that is perpendicular to the length of the channel defines the width of the channel. In some embodiments, the width of the media channel ranges from 20 μm to 100 μm, such as 30, 40, 50, 60, 70, 80 or 90 μm. The distance from the base of the media channel to the top of the walls defines the height of the media channel. In some embodiments, the height of the media channel ranges from 30 μm to 200 μm, such as 40, 50, 60, 70, 80, 90, 100, 110, 120, 130, 140, 150, 160, 170, 180 or 190 μm.

In some embodiments, a media channel is fluidly connected to a cell culture channel via a plurality of microchannels that are adapted to prevent cells from migrating between the cell culture channel and the media channel. As such, the microchannels have dimensions that allow fluid (e.g., cell culture medium) to pass through, but prevent the passage of cells. Each microchannel includes a base and two walls. In some embodiments, the height of each microchannel ranges from 0.1 μm to 5 μm, such as 0.5, 1, 1.5, 2, 2.5, 3, 3.5, 4, or 4.5 μm. In some embodiments, the width of each microchannel ranges from 0.1 μm to 5 μm, such as 0.5, 1, 1.5, 2, 2.5, 3, 3.5, 4, or 4.5 μm. The length of each microchannel is defined by the distance between the inner surface of the wall of the cell culture channel and the inner surface of the adjacent wall of the media channel. In some embodiments, the length of each microchannel ranges from 8 μm to 20 μm, such as 10, 12, 14, 16, or 18 μm. In certain embodiments, the length of each microchannel is 10 μm.

As used herein, the term “pitch” means the distance between two adjacent structures (e.g., two adjacent microchannels), as measured from the center of the first structure to the center of the second, adjacent structure. In some embodiments, the pitch of the microchannels ranges from 2 μm to 20 μm, such as 4, 6, 8, 10, 12, 14, 16, or 18 μm.

In some embodiments, a cell culture chamber includes a weir that is disposed near the outlet of the chamber and is configured to trap cells within the cell culture chamber while allowing fluid to pass. As such, the weir is configured or adapted to partially block the outlet of the cell culture chamber. The width of the weir is equal to the width of the cell culture chamber, such that the weir extends across the entire width of the cell culture chamber, or the outlet thereof. The height of the weir is less than the height of the cell culture chamber, such that in use, fluid is able to pass through a gap, or space between the bottom of the weir and the base of the cell culture chamber, while cells are retained within the cell culture chamber. In some embodiments, the difference between the height of the weir and the height of the cell culture chamber ranges from 1 μm to 5 μm, such as 1.5, 2, 2.5, 3, 3.5, 4 or 4.5 μm. In use, the weir provides for low pressure loading of cells into the cell culture chamber because fluid can pass through the gap between the weir and the base of the cell culture chamber, while cells are retained within the chamber. Fluid can pass through the gap under the weir and out through the outlet of the cell culture chamber without having to pass through, e.g., the microchannels between the cell culture chamber and the media channel(s); or the pores of a membrane that separates the cell culture chamber from the media channel(s). This configuration facilitates loading cells into the cell culture chamber at low pressure (e.g., a pressure ranging from 25 Pa to 75 Pa, such as 30, 35, 40, 45, 50, 55, 60, 65, or 70 Pa) by avoiding the increase of pressure associated with forcing fluid through the microchannels or through the membrane pores. In some embodiments, cells are loaded into the cell culture chamber at a pressure of 50 Pa using gravitational loading with a liquid height ranging from 0.2 cm to 0.8 cm, such as 0.5 cm.

Microfluidic cell culture units in accordance with embodiments of the invention are further described in PCT Patent Application No. PCT/US2014/047482, the disclosure of which is herein incorporated by reference in its entirety.

Microfluidic cell culture units in accordance with embodiments of the invention have a plurality of ports that are configured to allow the introduction and/or removal of fluids and/or cells from the unit. For example, in some embodiments, a microfluidic cell culture unit includes one or more ports that are configured to allow the introduction of a cell culture medium into the media channel(s) of the device. In some embodiments, a device includes a port that is configured to allow the introduction of a fluid that comprises cells into one or more cell culture chambers of the device. The ports are configured such that fluid connections can readily be established under sterile conditions, as desired, to add and/or remove fluids and/or cells from the device.

In some embodiments, a microfluidic cell culture unit includes a cell introduction port that provides access to the inlet end of the cell culture chamber. In use, this port is used to introduce cells into the cell culture chamber of the microfluidic cell culture unit. In some embodiments, a microfluidic cell culture unit includes a cell removal port that provides access to the cell culture chamber at or near the outlet end of the cell culture chamber. In use, this port is used to remove or extract cells from the cell culture chamber of the microfluidic cell culture unit.

In some embodiments, a microfluidic cell culture unit includes a media inlet port that provides common access to the inlet end of the media channel(s) of the microfluidic cell culture unit. In use, this port allows the introduction of a cell culture medium into all of the media channels of the microfluidic cell culture unit. In some embodiments, a microfluidic cell culture unit includes a media outlet port that provides common access to the outlet end of the media channel(s) of the microfluidic cell culture unit. In use, this port allows the collection of cell culture medium that has passed through the microfluidic cell culture unit. When cells are present in a cell culture chamber of the microfluidic cell culture unit, the cell culture medium that is collected from the media outlet port has been in fluid contact with the cells.

Microfluidic cell culture units in accordance with embodiments of the present disclosure can be made from any of a variety of suitable materials, including but not limited to elastomers (e.g., polydimethylsiloxane (PDMS)), thermosets (e.g., polyimide, polyurethane, SU-8), thermoplastics (e.g., polymethylmethacrylate (PMMA), polycarbonate (PC), polystyrene (PS), polyethylene terephthalate (PET) or polyvinylchloride (PVC)), polyesters (e.g., polycaprolactone (PCL)), or other materials, such as glass, quartz, or silicon. Combinations of two or more of the aforementioned materials can also be used.

In some embodiments, fabrication of a microfluidic cell culture unit is accomplished using multilayer photolithography and molding techniques. In some embodiments, a rigid mold is created using multilayer photolithography, and then the mold is used to cast a microfluidic cell culture unit in a suitable material, e.g., an elastomeric material, such as PDMS.

In some embodiments, a polyepoxide (epoxy) resin is used as a photoresist material in the mold fabrication process. In the mold fabrication process, a silicon wafer is cleaned with a mixture of 70% sulfuric acid and 30% hydrogen peroxide by volume, followed by a dehydration bake. The wafer is then spin-coated with a layer of photoresist material (e.g., SU8—2001 (MicroChem Corp, Mass., USA)) and subsequently soft-baked to evaporate residual solvents from the photoresist film. Then, the substrate is patterned via conventional UV photolithography. A chrome photomask with desired device features is formed for the first level of lithography. The photoresist is then exposed to UV light on a mask aligner (Karl Suss MA-6). After exposure, the wafer is postbaked on a hot plate and developed with a developer (SU-8 developer, MicroChem Corp, MA, USA). Next, the wafer is hard baked.

In some embodiments, the fabrication process includes a second level of photolithography to create additional features of the cell culture unit. For the second level of photolithography, the wafer is coated with another layer of photoresist and soft-baked on a hot plate. A second chrome photomask with desired device features is formed for the second level of photolithography. The photoresist is exposed to UV light on a mask aligner and post-exposure baked and/or developed with a developer as needed to create a photoresist mold that can be used to cast a microfluidic cell culture unit. In some embodiments, multiple levels of photolithography are used to create the mold, such as 2, 3, or 4 levels of photolithography. In some embodiments, a positive or a negative photoresist material may be utilized in any level of the photolithography process, as needed, to create a desired feature of the mold.

Following production of the microfluidic cell culture unit mold, the cell culture unit is cast in a suitable material. In some embodiments, the photoresist mold is contacted with a material that facilitates the release of the final material from the mold following the casting process. Examples of materials that facilitate the release of the final material from the mold include, but are not limited to, trichlorosilane (Gelest, Inc). To cast the cell culture unit in the mold, the final material, e.g., PDMS (Sylgard 184, Dow Corning) is mixed thoroughly with a curing agent in a suitable ratio (e.g., a ratio of 10:1) and degassed in a vacuum chamber to remove any trapped air. The mixture is then poured into the mold and cured at a designated temperature for a sufficient amount of time for the final material to cure. In some embodiments, the curing process is conducted at a temperature of 65° C. for a period of 12 hours. The final material is then removed from the mold. Additional features of the microfluidic cell culture unit can be added after the molding process has been completed. For example, in some embodiments, fluidic ports may be added to the unit by removing a portion of the material using a suitable instrument, such as, e.g., a biopsy punch (Harris Uni-Core).

Following molding and curing, a microfluidic cell culture unit is bonded to a flat sealing component to seal the unit. In some embodiments, the sealing component comprises a glass substrate. In some embodiments, the bonding process is facilitated by oxidizing the microfluidic cell culture unit and the sealing component in a suitable environment, such as an oxygen plasma environment, under suitable conditions. In some embodiments, oxidizing is conducted in an oxygen plasma environment for 20 seconds at 60 W, 10 atm cm³/min, and 20 mTorr.

In some embodiments, one or more surfaces of the microfluidic cell culture unit may be contacted with a compound that is adapted to promote adhesion of cells to the cell culture unit. Examples of compounds that promote adhesion of cells include, but are not limited to, proteins, such as, e.g., fibronectin, laminin, matrigel and collagen; and adhesion peptides, such as, e.g., bsp-RGD(15), AG-10 (CGGNRWHSIYITRFG; SEQ ID NO:2), AG-32 (CGGTWYKIAFQRNRK; SEQ ID NO:3), C-16 (CGGKAFDITYVRLKF; SEQ ID NO:4), or AG-73 (CGGRKRLQVQLSIRT; SEQ ID NO:5). In some embodiments, the compound that promotes adhesion of cells is placed in solution (e.g., in phosphate buffered saline (PBS)) and is incubated with the cell culture unit under suitable conditions for the compound to sufficiently adhere to the surface of the cell culture unit. In some embodiments, the compound that promotes adhesion of cells is deposited in a desired pattern on a surface of the cell culture unit in order to promote adhesion of cells in the desired pattern.

In some embodiments, one or more surfaces of a microfluidic cell culture unit may be modified to reduce or prevent adsorption and/or absorption of molecules. For example, in some embodiments, a microfluidic cell culture unit may be contacted with (e.g., coated with) one or more compositions that is configured to reduce adsorption of one or more molecules onto the surface. Examples of compositions that are configured to reduce adsorption include, but are not limited to, silanes, such as allylhydrodopolycarbosilane (AHPCS). In some embodiments, a microfluidic cell culture unit may be contacted with (e.g., coated with) one or more compositions that is configured to reduce the absorption of one or more molecules by the surface. Examples of compositions that are configured to reduce absorption include, but are not limited to, silica particles.

In some embodiments, a plurality of individual microfluidic cell culture units are fabricated on the same substrate, such that a single master chip contains multiple individual microfluidic cell culture units. In some embodiments, a single substrate includes 2, 4, 6, 8, 10, 12, 14, 16, 18, 20, 22, 24, 26, 28, 30, 32, 34, 36, 38, 40, 42, 44, 46, 48, or 50 or more, such as 60, 70, 80, 90 or 100 individual microfluidic cell culture units. In certain embodiments, the individual microfluidic cell culture units are arranged on a master chip such that features of the microfluidic cell culture units are evenly (e.g., uniformly, regularly) spaced on the master chip. For example, in certain embodiments, the media inlet and outlet ports of the individual microfluidic cell culture units are aligned on an equidistant grid, such that the spacing between the media inlet and outlet ports is the same across the entire substrate. In some embodiments, the distance between the evenly spaced media inlet and outlet ports ranges from 5 to 20 mm, such as 10 to 15 mm. In some embodiments, a single sealing component is configured to simultaneously seal a plurality of individual microfluidic cell culture units that are fabricated on the same master chip.

A master chip containing a plurality of individual microfluidic cell culture units can vary in size. In some embodiments, a master chip is comparable in size to a standard cell culture or tissue culture plate, e.g., has similar dimensions to a standard 96-well cell culture plate. In some embodiments, a master chip containing a plurality of individual microfluidic cell culture units has a length that ranges from 100 to 150 mm, such as 110, 120, 130 or 140 mm. In certain embodiments, a master chip containing a plurality of individual microfluidic cell culture units has a length that ranges from 127 to 128 mm.

In some embodiments, a master chip containing a plurality of individual microfluidic cell culture units has a width that ranges from 80 to 100 mm, such as 85, 90, or 95 mm. In certain embodiments, a master chip containing a plurality of individual microfluidic cell culture units has a width that ranges from 85 to 86 mm. In some embodiments, a master chip containing a plurality of individual microfluidic cell culture units has a height that ranges from 10 to 25 mm, such as 15 or 20 mm.

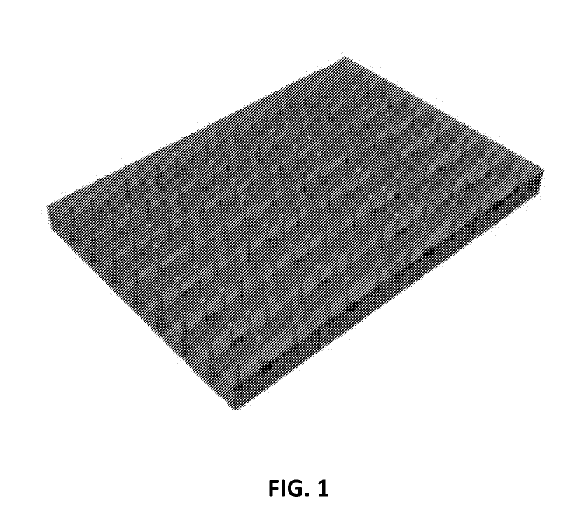

Referring now to FIG. 1, a master chip comprising a plurality of individual microfluidic cell culture units is shown. In the depicted embodiment, the media inlet and outlet ports of the microfluidic cell culture units are evenly spaced.

### Connectors

Aspects of the disclosure include connectors that are configured to fluidly connect two or more microfluidic cell culture units to one another. Connectors in accordance with embodiments of the invention can have any suitable dimensions to facilitate the formation of a fluid connection between two or more individual microfluidic cell culture units, as described above, and to facilitate a desired volumetric flow rate of cell culture medium from one microfluidic cell culture unit to another.

Connectors in accordance with embodiments of the invention include at least one inlet port and at least one outlet port that are connected by one or more channels. In use, the inlet port of a connector is fluidly connected to a media inlet or outlet port of a first microfluidic cell culture unit, and the outlet port of the connector is fluidly connected to a media inlet or outlet port of a second microfluidic cell culture unit, thereby establishing a fluid connection between the first and second microfluidic cell culture units.

Connectors in accordance with embodiments of the invention are configured to establish any of a variety of connection patterns between two or more individual microfluidic cell culture units. For example, in some embodiments, a connector is configured to connect two microfluidic cell culture units in series, whereas in some embodiments, a connector is configured to connect two or more microfluidic cell culture units in parallel. In some embodiments, a connector is configured to connect two or more microfluidic cell culture units in series, while also connecting two or more microfluidic cell culture units in parallel. In some embodiments, a connector includes 2 to 8 inlet ports, such as 3, 4, 5, 6, or 7 inlet ports. In some embodiments, a connector includes 2 to 8 outlet ports, such as 3, 4, 5, 6, or 7 outlet ports.

The inlet port(s) and outlet port(s) of a connector in accordance with embodiments of the invention are connected by one or more channels. The cross sectional area of the channel(s) of a connector can be varied, as desired, to achieve a desired flow of liquid through the connector. For example, in some embodiments, a channel connecting an inlet port of a connector to an outlet port of a connector has a width ranging from 30 to 100 μm, such as 40, 50, 60, 70, 80 or 90 μm, and has a height ranging from 20 to 100 μm, such as 30, 40, 50, 60, 70, 80 or 90 μm. In some embodiments, the length of a channel connecting an inlet port of a connector to an outlet port of a connector is a multiple of the spacing distance between the evenly spaced media inlet and outlet ports of the microfluidic cell culture units. A channel that connects an inlet port of a connector to an outlet port of a connector can be straight (e.g., can extend in a single direction along its entire length), or can have any number of bends or turns. For example, in some embodiments, a channel that connects an inlet port of a connector to an outlet port of a connector can have a plurality of 90° turns, such as 1, 2, 3, 4, 5, 6, 7, or 8 or more 90° turns along its length.

In some embodiments, a connector comprises two or more channels that connect the inlet port(s) to the outlet port(s). In certain embodiments, the channels have the same dimensions, e.g., the channels are the same height, width and length. In some embodiments, the channels have different dimensions. For example, in some embodiments a first channel is shorter in length than a second channel. In some embodiments, a first channel has a different cross sectional area, e.g., is shorter in height and/or has a smaller width, as compared to a second channel.

Connectors in accordance with embodiments of the invention can be made from any of a variety of suitable materials, including but not limited to elastomers (e.g., polydimethylsiloxane (PDMS)), thermosets (e.g., polyimide, polyurethane, SU-8), thermoplastics (e.g., polymethylmethacrylate (PMMA), polycarbonate (PC), polystyrene (PS), polyethylene terephthalate (PET) or polyvinylchloride (PVC)), polyethylene or copolymers thereof, polypropylene or copolymers thereof, or other materials, such as glass, quartz, or silicon. Combinations of two or more of the aforementioned materials can also be used. In some embodiments, fabrication of a connector is accomplished using multilayer photolithography and molding techniques, as described above. In some embodiments, a rigid mold is created using photolithography techniques, and then the mold is used to cast a connector in a suitable material, e.g., an elastomeric material, such as PDMS. Following molding and curing, a connector is bonded to a flat sealing component to seal the connector. In some embodiments, the sealing component comprises a glass substrate. In some embodiments, the bonding process is facilitated by oxidizing the connector and the sealing component in a suitable environment, such as an oxygen plasma environment, under suitable conditions. In some embodiments, oxidizing is conducted in an oxygen plasma environment for 20 seconds at 60 W, 10 atm cm³/min, and 20 mTorr.

Connectors in accordance with some embodiments of the invention are three dimensional structures that can be “plugged in” to a master chip that comprises a plurality of individual microfluidic cell culture units, as described above. For example, in some embodiments, a connector includes a solid block of material that comprises a channel, as described above. In some embodiments, a tube having a length that ranges from 15 to 45 mm, such as 20, 25, 30, 35, or 40 mm, is inserted into and/or bonded to the inlet and outlet ports of the connector. The inner diameter of a tube in accordance with embodiments of the invention can be varied so as to minimize the dead volume within the tube, as well as the media travel time within the tube, while also meeting the other functional requirements of the system. In certain embodiments, a tube has an internal diameter that ranges from 50 to 2,000 μm, such as 250, 500, 750, 1,000, 1,250, 1,500, or 1,750 μm. Tubes in accordance with embodiments of the invention may include any suitable material, such as metal, plastic, ceramic, or any combination thereof. In some embodiments, a tube is made from stainless steel. In some embodiments, a tube is made of glass or quartz.

In some embodiments, the tubes that are inserted into and/or bonded to the inlet and outlet ports of the connector are oriented so as to be substantially perpendicular to the direction of the channel that connects the inlet and outlet ports of the connector. In use, a connector can be positioned above the master chip that contains a plurality of individual microfluidic cell culture units, and the tubes can be “plugged in” to a desired media inlet port and media outlet port of one or more individual microfluidic cell culture units on the master chip. As such, a fluid connection is formed between the first and second microfluidic cell culture units via the connector. In some embodiments, a plurality of connectors can be plugged into the master chip to form a desired connection pattern between the individual microfluidic cell culture units on the master chip.

In some embodiments, a connector comprises an auxiliary port that provides access to the channel that extends from the inlet port(s) of the connector to the outlet port(s) of the connector. The auxiliary port can be used to introduce and/or remove a liquid, such as, e.g., a cell culture medium, directly into or from the connector. In certain embodiments, a connector comprises a sensor that is configured to measure a characteristic of the cell culture medium passing through the connector. Characteristics of the cell culture medium that can be measured using a sensor include, but are not limited to, the concentration of one or more components of the cell culture medium, e.g., a glucose level or an oxygen level, a pH, and the like. In some embodiments, a connector may comprise an electrode that is configured to measure an electrochemical characteristic of the cell culture medium.

In some embodiments, a connector may include one or more sensors that are configured to measure one or more characteristics of a cell culture medium. Examples of characteristics of the cell culture medium that can be measured by the sensor include, but are not limited to, pH, dissolved oxygen, and concentration of various molecules (e.g., concentration of glucose, lactate, albumin, or fatty acids) in the cell culture medium.

Sensors in accordance with embodiments of the invention can be incorporated into a connector using any of a variety of suitable techniques. For example, in some embodiments, a sensor may be fluidly coupled to a connector so that a fluid moving through the connector contacts that sensor. In some embodiments, a sensor may be fabricated in a substrate (e.g., a sensor may be patterned in a surface using photolithography and/or chemical vapor deposition techniques), and a connector is fluidly connected to the substrate so that a fluid moving through the connector contacts the sensor.

In certain embodiments, a sensor may include one or more electrodes that are configured to measure one or more characteristics of a cell culture medium. Electrodes in accordance with embodiments of the invention may include any conductive material, including but not limited to, gold, silver, tin oxide, indium tin oxide (ITO) or platinum. In some embodiments, an electrode may be deposited on a base layer of a suitable material, such as, e.g., glass, silicon, or polyethylene terephthalate (PET).

In some embodiments, an electrode may be functionalized with one or more compositions that are configured to facilitate the detection of a target molecule by the electrode. For example, in some embodiments, an electrode may be functionalized with an enzyme (e.g., a glucose oxidase enzyme (GOx)) that is configured to generate a detectable chemical composition in the presence of a target molecule. Electrode functionalization may be accomplished using any suitable techniques for stably associating a composition with the surface of an electrode. For example, in some embodiments, an enzyme may be stably associated with an electrode surface by attaching the enzyme to the electrode surface with a linking molecule (e.g., a thiol linker molecule) that is configured to facilitate that stable association of the enzyme with the electrode surface.

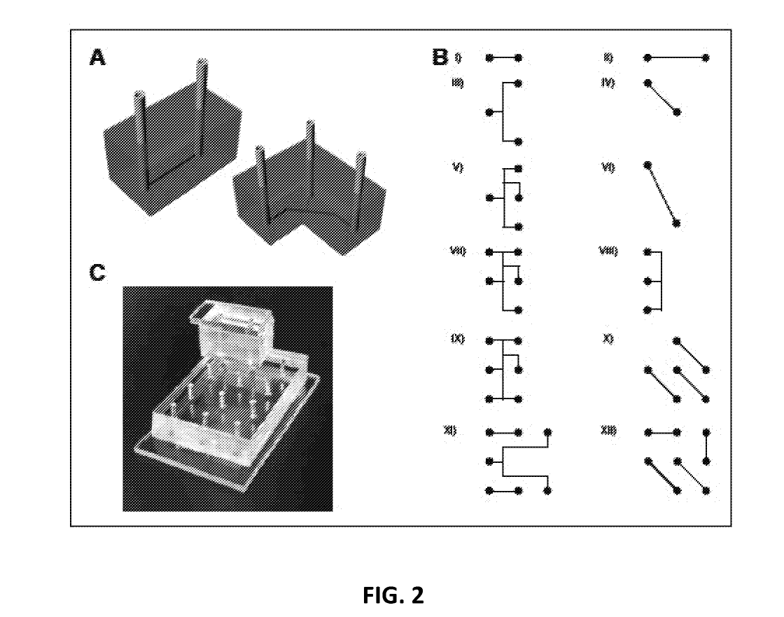

Referring now to FIG. 2, several different connectors are shown. Panel A depicts two different connectors. The first connector is configured to connect a first and a second microfluidic cell culture unit in series. The second connector is configured to connect a first microfluidic cell culture unit in parallel to two different microfluidic cell culture units.

Panel B depicts various different channel geometries that can be used to create complex and customizable circulation patterns between microfluidic cell culture units. Panel C depicts a master chip comprising a plurality of individual microfluidic cell culture units, and also shows a connector plugged into the master chip to connect a first and a second microfluidic cell culture unit in series.

### Computer Programs

Aspects of the subject systems include a controller, a processor and a computer readable medium that are configured or adapted to operate one or more components of the subject systems and/or devices. In some embodiments, a system includes a controller that is in communication with one or more components of the devices or systems, as described herein, and is configured to control aspects of the devices or systems and/or execute one or more operations or functions of the subject devices or systems. In some embodiments, a system includes a processor and a computer-readable medium, which may include memory media and/or storage media. Applications and/or operating systems embodied as computer-readable instructions on computer-readable memory can be executed by the processor to provide some or all of the functionalities described herein.

In some embodiments, a system includes a user interface, such as a graphical user interface (GUI), that is adapted or configured to receive input from a user, and to execute one or more of the methods as described herein. In some embodiments, a GUI is configured to display data or information to a user.

### Volumetric Flow Rate Control Systems

Aspects of the disclosure include additional components that can be used in conjunction with the subject microfluidic cell culture units and connectors, as described above. For example, in some embodiments, the subject systems include pumps, valves, mass flow controllers, reservoirs, sterile filters, syringes, pipettes, and/or any other fluid handling devices or components. In some embodiments, a subject system includes a volumetric flow rate control system that is configured to control the volumetric flow rate of a cell culture medium that passes from a first microfluidic cell culture unit to another microfluidic cell culture unit. By “volumetric flow rate” is meant a volume of fluid that passes per unit time, e.g., μL/s.

In some embodiments, the subject systems include a volumetric flow rate control system that includes a library of organ-specific parameters. Organ-specific parameters may include, e.g., a fluid constituent consumption parameter, a fluid storage parameter, a fluid volume to tissue volume ration, and/or a fluid resistance property that are representative of a particular organ or tissue that is modeled by one or more microfluidic cell culture units. In some embodiments, the volumetric flow rate control system is used to control the flow of a cell culture medium between at least two microfluidic cell culture units in order to replicate or model a natural circulation of bodily fluid between two or more organs or organ-systems in a subject.

In certain embodiments, a volumetric flow rate control system is configured to receive one or more user inputs, such as, e.g., information regarding the number and/or type of connector(s) that are used to connect two or more different microfluidic cell culture units, the number and/or type of cells that are cultured in the microfluidic cell culture units, or the like. In some embodiments, a volumetric flow rate control system is configured to receive one or more user inputs that include, e.g., one or more organ-specific parameters that are to be applied to the system in order to mimic the natural circulation of a bodily fluid between two or more organs or tissues in a subject. In some embodiments, a volumetric flow rate control system is configured to receive a user input in the form of a specific volumetric flow rate, e.g., a flow rate ranging from 10 μL/hour up to 5 mL/hour, such as 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950 or 975 μL/h or more, such as 1.0, 1.25, 1.5, 1.75, 2.0, 2.25, 2.5, 2.75, 3.0, 3.25, 3.5, 3.75, 4.0, 4.25, 4.5, or 4.75 mL/hour. In use, the volumetric flow rate control system applies the user input to control the flow of a cell culture medium between the first and second microfluidic cell culture units.

### Cell Culture Medium

Aspects of the disclosure include a cell culture medium that is configured or adapted to support the growth and maintenance of a plurality of cells that are cultured within the subject microfluidic cell culture units. In some embodiments, a universal cell culture medium is configured to support a plurality of different cell types. For example, in certain embodiments, a universal cell culture medium is circulated through each of the microfluidic cell culture units, wherein each microfluidic cell culture unit contains a different cell type, and the same universal cell culture medium supports growth and/or maintenance of each different cell type.

In some embodiments, a cell culture medium includes one or more of the following components: a standard mammalian cell culture minimal medium, which may include a high glucose concentration; sodium pyruvate; a vitamin (e.g., B27); a differentiation factor; and a growth factor. In some embodiments, a cell culture medium includes the following components: a standard mammalian cell culture minimal medium, which may include a high glucose concentration; sodium pyruvate; a vitamin (e.g., B27); and a growth factor.

Examples of suitable growth factors include, but are not limited to, oncostatin M; hepatocyte growth factor; vascular endothelial growth factor; 6kine, activin A, amphiregulin, angiogenin, β-endothelial cell growth factor, β-cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocyte-macrophage colony stimulating factor, GRO-α/MGSA, GRO-β, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, mechano growth factor (MGF), insulin-like growth factor II, nerve growth factor, neurotophin-3,4, oncostatin M, placenta growth factor, pleiotrophin, rantes, stem cell factor, stromal cell-derived factor 1B, thrombopoietin, transforming growth factor-(alpha, beta1,2,3,4,5), tumor necrosis factor (alpha and beta), vascular endothelial growth factors, and bone morphogenic proteins. Growth factors in accordance with embodiments of the invention may be monovalent or multivalent.

Any of a variety of standard cell culture media can be used. In some embodiments, a cell culture medium is a conditioned medium that has previously been contacted with one or more different cell types. For example, in some embodiments, a cell culture medium has previously been contacted with, e.g., endothelial cells, fibroblast cells (e.g., 3T3-J2 cells), or a similar cell type. In certain embodiments, one or more different cell types can be directly incorporated into a cell culture chamber and cultured therein in contact with a cell culture medium in order to produce a conditioned medium.

### Cells

Cells that can be cultured in a microfluidic device of the present disclosure include stem cells; induced pluripotent stem (iPS) cells; human embryonic stem (hES) cells; mesenchymal stem cells (MSCs); multipotent progenitor cells; cardiomyocytes; cardiomyocyte progenitors; hepatocytes; beta islet cells; neurons, e.g., astrocytes, neuronal sub-populations; leukocytes; endothelial cells; lung epithelial cells; exocrine secretory epithelial cells; hormone-secreting cells, such as anterior pituitary cells, magnocellular neurosecretory cells, thyroid epithelial cells, adrenal gland cells, etc.; keratinocytes; lymphocytes; macrophages; monocytes; renal cells; urethral cells; sensory transducer cells; autonomic neuronal cells; central nervous system neurons; glial cells; skeletal muscle cells; a kidney cell, e.g., a kidney parietal cell, a kidney glomerulus podocyte, etc.; white adipocytes (e.g., white adipose tissue (WAT)), brown adipocytes; adipose-derived stem cells; osteocytes; osteoblasts; chondrocytes; smooth muscle cells; microglial cells; stromal cells; etc. In some embodiments, a cell is genetically modified to express a reporter polypeptide.

In some embodiments, stem cells or progenitor cells that have been differentiated into cells of one or more specific organs or tissues are cultured in the subject microfluidic devices. In certain embodiments, a stem cell or progenitor cell is initially cultured in a subject microfluidic device, and the stem cell or progenitor cell is then differentiated into a specific cell type.

In some cases, cells cultured in a microfluidics device of the present disclosure are healthy. In some cases, cells cultured in a microfluidics device of the present disclosure are diseased. In some cases, cells cultured in a microfluidics device of the present disclosure include one or more genetic mutations that pre-dispose the cells to disease. Both non-cancerous as well as cancerous cells can be cultured in the subject microfluidic devices. In some embodiments, cells from a cancer cell line are cultured in the subject microfluidic devices. In certain embodiments, cells from a breast cancer cell line are cultured in the subject microfluidic devices.

In some cases, the cells cultured in a device or system of the present disclosure are primary cells. In some cases, the cells cultured in a device or system of the present disclosure are primary cells obtained from a healthy individual. In some cases, the cells cultured in a device or system of the present disclosure are primary cells obtained from a diseased individual. In some cases, the cells cultured in a device or system of the present disclosure are obtained from an individual who has a disease-associated mutation, but who has not been diagnosed as having a disease associated with the disease-associated mutation. In some cases, the cells cultured in a device or system of the present disclosure are all obtained from a single individual. In some cases, the cells cultured in a device or system of the present disclosure are obtained from two or more different individuals.

In some cases, the cells cultured in a device or system of the present disclosure are human cells. In some cases, the cells cultured in a device or system of the present disclosure are non-human mammalian cells. In some cases, the cells cultured in a device or system of the present disclosure are rat cells. In some cases, the cells cultured in a device or system of the present disclosure are mouse cells. In some cases, the cells cultured in a device or system of the present disclosure are pig cells. In some cases, the cells cultured in a device or system of the present disclosure are non-human primate cells.

In some cases, 2, 3, 4, 5, 6, 7, 8, 9, 10, or more than 10, different cell types are cultured in a device or system of the present disclosure. For example, in some cases, cardiomyocytes and hepatocytes are cultured in a device or system of the present disclosure.

### Cardiomyocytes

In some cases, cells that are cultured in a microfluidic device of the present disclosure are cardiomyocytes. The following discussion as it relates to cardiomyocytes is applicable to any of a variety of cell types, as described above, which may be cultured in a subject microfluidic device. The following discussion of cardiomyocytes is therefore exemplary and not intended to be limiting.

Cells that can be cultured in a microfluidic device of the present disclosure include cardiomyocytes, cardiomyocyte progenitors, induced pluripotent stem (iPS) cells, and the like. In some cases, the cardiomyocytes or cardiomyocyte progenitors are healthy cardiomyocytes or cardiomyocyte progenitors. In some cases, the cardiomyocytes or cardiomyocyte progenitors are diseased cardiomyocytes or cardiomyocyte progenitors. For example, in some cases, the cardiomyocytes or cardiomyocyte progenitors are from an individual having a cardiovascular disease or condition. For example, in some cases, the cardiomyocytes or cardiomyocyte progenitors are from an individual having an ischemic heart disease, an arrhythmia, tachycardia, bradycardia, myocardial infarction, or a congenital heart condition. For example, in some cases, the cardiomyocytes or cardiomyocyte progenitors are from an individual having long QT syndrome (LQTS). Congenital LQTS is an inherited cardiac arrhythmic disease that results from ion channel defects. Drug-induced LQTS can be acquired following use of certain pharmaceutical agents. In some embodiments, human cardiac myocyte (HCM) cells are cultured in the subject microfluidic devices. In some embodiments, dilated cardiomyopathy (DCM) cells are cultured in the subject microfluidic devices.

Cells that can be cultured in a microfluidics device of the present disclosure include induced pluripotent stem cells (iPS cells). In some cases, the iPS cells are generated from somatic cells obtained from healthy individuals. In some cases, the iPS cells are generated from somatic cells obtained from individuals having a cardiovascular disease or condition. For example, in some cases, the iPS cells are generated from a somatic cell obtained from an individual having a cardiovascular disease or condition such as ischemic heart disease, arrhythmia, tachycardia, bradycardia, myocardial infarction, or a congenital heart condition.

Cardiomyocytes can have certain morphological characteristics. They can be spindle, round, triangular or multi-angular shaped, and they may show striations characteristic of sarcomeric structures detectable by immunostaining. They may form flattened sheets of cells, or aggregates that stay attached to the substrate or float in suspension, showing typical sarcomeres and atrial granules when examined by electron microscopy

Cardiomyocytes and cardiomyocyte precursors generally express one or more cardiomyocyte-specific markers. Cardiomyocyte-specific markers include, but are not limited to, cardiac troponin I (cTnI), cardiac troponin-C, cardiac troponin T (cTnT), tropomyosin, caveolin-3, myosin heavy chain (MHC), myosin light chain-2a, myosin light chain-2v, ryanodine receptor, sarcomeric α-actinin, Nkx2.5, connexin 43, and atrial natriuretic factor (ANF). Cardiomyocytes can also exhibit sarcomeric structures. Cardiomyocytes exhibit increased expression of cardiomyocyte-specific genes ACTC1 (cardiac α-actin), ACTN2 (actinin a2), MYH6 (α-myosin heavy chain), RYR2 (ryanodine receptor 2), MYL2 (myosin regulatory light chain 2, ventricular isoform), MYL7 (myosin regulatory light chain, atrial isoform), TNNT2 (troponin T type 2, cardiac), and NPPA (natriuretic peptide precursor type A), PLN (phospholamban).

In some cases, cardiomyocytes can express cTnI, cTnT, Nkx2.5; and can also express at least 3, 4, 5, or more than 5, of the following: ANF, MHC, titin, tropomyosin, α-sarcomeric actinin, desmin, GATA-4, MEF-2A, MEF-2B, MEF-2C, MEF-2D, N-cadherin, connexin-43, β-1-adrenoreceptor, creatine kinase MB, myoglobin, α-cardiac actin, early growth response-I, and cyclin D2.

In some cases, a cardiomyocyte is generated from an iPS cell, where the iPS cell is generated from a somatic cell obtained from an individual.

### Patient-Specific Cells

In some cases, the cells are patient-specific cells. In some cases, the patient-specific cells are derived from stem cells obtained from a patient. In some cases, the patient-specific cells are derived from iPS cells generated from somatic cells obtained from a patient. In some cases, patient-specific cells are primary cells. In some cases, the cells form embryoid bodies (EBs).

Suitable stem cells include embryonic stem cells, adult stem cells, and induced pluripotent stem (iPS) cells.

iPS cells are generated from mammalian cells (including mammalian somatic cells) using, e.g., known methods. Examples of suitable mammalian cells include, but are not limited to: fibroblasts, skin fibroblasts, dermal fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, epithelial cells, gastric epithelial cells, lung epithelial cells, synovial cells, kidney cells, skin epithelial cells, pancreatic beta cells, and osteoblasts.

Mammalian cells used to generate iPS cells can originate from a variety of types of tissue including but not limited to: bone marrow, skin (e.g., dermis, epidermis), muscle, adipose tissue, peripheral blood, foreskin, skeletal muscle, and smooth muscle. The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

iPS cells produce and express on their cell surface one or more of the following cell surface antigens: SSEA-3, SSEA-4, TRA-1-60, TRA-1-81, TRA-2-49/6E (alkaline phophatase), and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT.

Methods of generating iPS cells are known in the art, and a wide range of methods can be used to generate iPS cells. See, e.g., Takahashi and Yamanaka (2006) Cell 126:663-676; Yamanaka et al. (2007) Nature 448:313-7; Wernig et al. (2007) Nature 448:318-24; Maherali (2007) Cell Stem Cell 1:55-70; Maherali and Hochedlinger (2008) Cell Stem Cell 3:595-605; Park et al. (2008) Cell 134:1-10; Dimos et. al. (2008) Science 321:1218-1221; Blelloch et al. (2007) Cell Stem Cell 1:245-247; Stadtfeld et al. (2008) Science 322:945-949; Stadtfeld et al. (2008) 2:230-240; Okita et al. (2008) Science 322:949-953.

In some embodiments, iPS cells are generated from somatic cells by forcing expression of a set of factors in order to promote increased potency of a cell or de-differentiation. Forcing expression can include introducing expression vectors encoding polypeptides of interest into cells, introducing exogenous purified polypeptides of interest into cells, or contacting cells with a reagent that induces expression of an endogenous gene encoding a polypeptide of interest.

Forcing expression may include introducing expression vectors into somatic cells via use of moloney-based retroviruses (e.g., MLV), lentiviruses (e.g., HIV), adenoviruses, protein transduction, transient transfection, or protein transduction. In some embodiments, the moloney-based retroviruses or HIV-based lentiviruses are pseudotyped with envelope from another virus, e.g. vesicular stomatitis virus g (VSV-g) using known methods in the art. See, e.g. Dimos et al. (2008) Science 321:1218-1221.

In some embodiments, iPS cells are generated from somatic cells by forcing expression of Oct-3/4 and Sox2 polypeptides. In some embodiments, iPS cells are generated from somatic cells by forcing expression of Oct-3/4, Sox2 and Klf4 polypeptides. In some embodiments, iPS cells are generated from somatic cells by forcing expression of Oct-3/4, Sox2, Klf4 and c-Myc polypeptides. In some embodiments, iPS cells are generated from somatic cells by forcing expression of Oct-4, Sox2, Nanog, and LIN28 polypeptides.

For example, iPS cells can be generated from somatic cells by genetically modifying the somatic cells with one or more expression constructs encoding Oct-3/4 and Sox2. As another example, iPS cells can be generated from somatic cells by genetically modifying the somatic cells with one or more expression constructs comprising nucleotide sequences encoding Oct-3/4, Sox2, c-myc, and Klf4. As another example, iPS cells can be generated from somatic cells by genetically modifying the somatic cells with one or more expression constructs comprising nucleotide sequences encoding Oct-4, Sox2, Nanog, and LIN28.

In some embodiments, cells undergoing induction of pluripotency as described above, to generate iPS cells, are contacted with additional factors which can be added to the culture system, e.g., included as additives in the culture medium. Examples of such additional factors include, but are not limited to: histone deacetylase (HDAC) inhibitors, see, e.g. Huangfu et al. (2008) Nature Biotechnol. 26:795-797; Huangfu et al. (2008) Nature Biotechnol. 26: 1269-1275; DNA demethylating agents, see, e.g., Mikkelson et al (2008) Nature 454, 49-55; histone methyltransferase inhibitors, see, e.g., Shi et al. (2008) Cell Stem Cell 2:525-528; L-type calcium channel agonists, see, e.g., Shi et al. (2008) 3:568-574; Wnt3a, see, e.g., Marson et al. (2008) Cell 134:521-533; and siRNA, see, e.g., Zhao et al. (2008) Cell Stem Cell 3: 475-479.

In some embodiments, iPS cells are generated from somatic cells by forcing expression of Oct3/4, Sox2 and contacting the cells with an HDAC inhibitor, e.g., valproic acid. See, e.g., Huangfu et al. (2008) Nature Biotechnol. 26: 1269-1275. In some embodiments, iPS cells are generated from somatic cells by forcing expression of Oct3/4, Sox2, and Klf4 and contacting the cells with an HDAC inhibitor, e.g., valproic acid. See, e.g., Huangfu et al. (2008) Nature Biotechnol. 26:795-797.

Cardiomyocytes (e.g., patient-specific cardiomyocytes) can be generated from iPS cells using any known method. See, e.g., Mummery et al. (2012) Circ. Res. 111:344.

Under appropriate circumstances, iPS cell-derived cardiomyocytes often show spontaneous periodic contractile activity. This means that when they are cultured in a suitable tissue culture environment with an appropriate Ca²⁺ concentration and electrolyte balance, the cells can be observed to contract across one axis of the cell, and then release from contraction, without having to add any additional components to the culture medium. The contractions are periodic, which means that they repeat on a regular or irregular basis, at a frequency between about 6 and 200 contractions per minute, and often between about 20 and about 90 contractions per minute in normal buffer. Individual cells may show spontaneous periodic contractile activity on their own, or they may show spontaneous periodic contractile activity in concert with neighboring cells in a tissue, cell aggregate, or cultured cell mass.

Generation of Cardiomyocytes from iPSCs

Cardiomyocytes can be generated from iPSCs, or other stem cells, using well-known methods/See, e.g., Mummery et al. (2012) Circ. Res. 111:344; Lian et al. (2012) Proc. Natl. Acad. Sci. USA 109:E1848; Ye et al. (2013) PLoSOne 8:e53764.

Generation of Cardiomyocytes Directly from a Post-Natal Somatic Cell

A cardiomyocyte can be generated directly from a post-natal somatic cell, without formation of an iPS cell as an intermediate. For example, in some cases, a human post-natal fibroblast is induced directly (to become a cardiomyocyte, using a method as described in WO 2014/033123. For example, reprogramming factors Gata4, Mef2c, Tbx5, Mesp1, and Essrg are introduced into a human post-natal fibroblast to induce the human post-natal fibroblast to become a cardiomyocyte. In some cases, the polypeptides themselves are introduced into the post-natal fibroblast. In other cases, the post-natal fibroblast is genetically modified with one or more nucleic acids comprising nucleotide sequences encoding Gata4, Mef2c, Tbx5, Mesp1, and Essrg.

### Isogenic Pairs of Cardiomyocytes

In some cases, isogenic pairs of cardiomyocytes are used. In some cases, isogenic pairs of wild-type and genetically modified cardiomyocytes are used. In some cases, isogenic pairs of diseased and non-diseased cardiomyocytes are used. For example, in some cases, isogenic pairs of cardiomyocytes from an individual are used, where one of the isogenic pair is genetically modified with a nucleic acid comprising a nucleotide sequence encoding a mutant form of a polypeptide such that the genetically modified cardiomyocyte exhibits characteristics of a diseased cardiomyocyte.

In some cases, isogenic pairs of iPS cells are used. In some cases, isogenic pairs of wild-type and genetically modified iPS cells are used. In some cases, isogenic pairs of diseased and non-diseased iPS cells are used.

### Genetic Modification

In some cases, a cell cultured in a subject microfluidic device is genetically modified. For example, a cell can be genetically altered to express one or more growth factors of various types, such as FGF, cardiotropic factors such as atrial natriuretic factor, cripto, and cardiac transcription regulation factors, such as GATA-4, Nkx2.5, and MEF2-C. Genetic modification generally involves introducing into the cell a nucleic acid comprising a nucleotide sequence encoding a polypeptide of interest. The nucleotide sequence encoding the polypeptide of interest can be operably linked to a transcriptional control element, such as a promoter. Suitable promoters include, e.g., promoters of cardiac troponin I (cTnI), cardiac troponin T (cTnT), sarcomeric myosin heavy chain (MHC), GATA-4, Nkx2.5, N-cadherin, .beta.1-adrenoceptor, ANF, the MEF-2 family of transcription factors, creatine kinase MB (CK-MB), myoglobin, or atrial natriuretic factor (ANF).

In some cases, a cardiomyocyte is genetically modified with a nucleic acid comprising a nucleotide sequence encoding a mutant form of a polypeptide such that the genetically modified cardiomyocyte exhibits characteristics of a diseased cardiomyocyte. For example, a cardiomyocyte can be genetically modified to express a KVLQT1, HERG, SCN5A, KCNE1, or KCNE2 polypeptide comprising a mutation associated with LQTS, where the genetically modified cardiomyocyte exhibits characteristics associated with LQTS. See, e.g., Splawski et al. (2000) Circulation 102:1178, for mutations in KVLQT1, HERG, SCN5A, KCNE1, and KCNE2 that are associated with LQTS. For example, a cardiomyocyte can be genetically modified such that a gene encoding a KVLQT1, HERG, SCN5A, KCNE1, or KCNE2 polypeptide with a LQTS-associated mutation replaces a wild-type KVLQT1, HERG, SCN5A, KCNE1, or KCNE2 gene.

In some cases, a cell to be cultured in a subject microfluidic device is genetically modified to express one or more polypeptides that provide real-time detection of a cellular response. Such polypeptides include, e.g., calcium indicators, genetically encoded voltage indicators (GEVI; e.g., voltage-sensitive fluorescent proteins), sodium channel protein activity indicators, indicators of oxidation/reduction status within the cell, etc. For example, a cell can be genetically modified to include an indicator of Cyp3A4 activity.

In some cases, a cell (e.g., a cardiomyocyte or other cell) is genetically modified to express a genetically-encoded calcium indicator (GECI). See, e.g., Mank and Griesbeck (2008) Chem. Rev. 108:1550; Nakai et al. (2001) Nat. Biotechnol. 19:137; Akerboom et al. (2012) J. Neurosci. 32:13819; Akerboom et al. (2013) Front. Mol. Neurosci. 6:2. Suitable GECI include pericams, cameleons (Miyawaki et al (1999) Proc. Natl. Acad. Sci. USA 96:2135), and GCaMP. As one non-limiting example, a suitable GECI can be a fusion of a circularly permuted variant of enhanced green fluorescent protein (cpEGFP) with the calcium-binding protein calmodulin (CaM) at the C terminus and a CaM-binding M13 peptide (from myosin light chain) at the N terminus. Nakai et al. (2001) Nat. Biotechnol. 19:137. In some cases, a suitable GECI can comprise an amino acid sequence having at least 85%, at least 90%, at least 95%, at least 98%, or 100%, amino acid sequence identity with the following GCaMP6 amino acid sequence:

(SEQ ID NO: 1)
mgshhhhhhg masmtggqqm grdlyddddk dlatmvdssr
rkwnktghav raigrlssle nvyikadkqk ngikanfkir
hniedggvql ayhyqqntpi gdgpvllpdn hylsvqskls
kdpnekrdhm vllefvtaag itlgmdelyk ggtggsmvsk
geelftgvvp ilveldgdvn ghkfsysgeg egdatygklt
lkficttgkl pvpwptivtt lxvqcfsryp dhmkqhdffk
sampegyiqe rtiffkddgn yktraevkfe gdtlvnriel
kgidfkedgn ilghkleynl pdqlteeqia efkeafslfd
kdgdgtittk elgtvmrslg qnpteaelqd minevdadgd
gtidfpeflt mmarkgsyrd teeeireafg vfdkdgngyi
saaelrhvmt nlgekltdee vdemireadi dgdgqvnyee
fvqmmtak

### Methods of Culturing Cells

Aspects of the disclosure include methods for culturing cells using the subject devices and systems. In some embodiments, the methods involve introducing a plurality of cells into the cell culture channel of a microfluidic cell culture unit, and introducing a cell culture medium into the media channel(s) of the cell culture unit. Once the cells have been introduced into the cell culture unit, the subject methods involve maintaining the device under suitable cell culture conditions. In some embodiments, the cell culture conditions include a controlled temperature that ranges from 30° C. to 40° C., such as from 35° C. to 38° C. In some embodiments, the cell culture conditions include a controlled CO₂ gas concentration ranging from 2% to 10%, such as 4% to 6%. In some embodiments, the cell culture conditions include a controlled humidity environment to reduce evaporative loss of the cell culture medium.

In some embodiments, the subject methods involve moving a cell culture medium from a first microfluidic cell culture unit to a second microfluidic cell culture unit through a connector at a specified volumetric flow rate. In some embodiments, the cell culture medium is moved using gravity or using applied positive or negative pressure. In certain embodiments, the cell culture medium is a universal cell culture medium that is configured to support the growth and/or maintenance of a plurality of different cell types.

In some embodiments, the methods involve introducing a plurality of cells and a cell culture medium into the device, as described above, and maintaining the device under suitable cell culture conditions for a period of time that ranges from one day to one month. In certain embodiments, the methods involve removing a plurality of cells from the device after a specified period of time has elapsed. For example, in some embodiments, a plurality of cells may be cultured in the device for a period of time ranging from one day to one month, and the cells may then be removed from the device.

In some embodiments, the methods involve collecting data from the cells in the device during the culture process using one or more sensors. Data may be collected at any desired point in time during the culture process. In some embodiments, data may be collected at regular intervals during the culture process, e.g., may be collected on an hourly or a daily basis.

In some embodiments, the methods involve simulating the natural circulation between two or more different organs or tissues in a subject. For example, in certain embodiments, the subject methods involve culturing two or more different cell types, each in a different microfluidic cell culture unit of the device, connecting the different microfluidic cell culture units with one or more connectors, and applying a specified volumetric flow rate of a cell culture medium between the microfluidic cell culture units to simulate the natural circulation of body fluid between two or more different organs or tissues in a subject.

In certain embodiments, the subject methods involve stimulating a first plurality of cells with a stimulus, such as, e.g., a candidate active agent (e.g., a protein, or a pharmaceutical compound) or a virus (e.g., a hepatitis C virus) and measuring or determining the impact of the stimulus on a second plurality of cells that are in fluid communication with the first plurality of cells.

### Utility

The subject devices, systems and methods are useful for a variety of applications, including, but not limited to, drug screening; determining the potential effect of a drug on an individual; drug toxicity testing; disease modeling; and research applications, such as characterization of patient-specific cell populations.

### Research Applications

The kinetics of drug metabolism can be studied in real time using a device and system of the present disclosure. The effect of a test agent, which may be a known drug, or an agent not currently used as a drug, can be tested on multiple cell types using a multi-organ device and system of the present disclosure. Pharmacodynamic and pharmacokinetic properties of a test agent can be determined using a device and system of the present disclosure.

The device can include a built-in microscopic imaging system and/or a built-in stereoscopic imaging system to allow for the monitoring of cells in response to a test agent or other stimulus. Chemical transformation of a test agent and/or consumption of a test agent can be monitored using a multi-organ device and system of the present disclosure. In some cases, a measure of electrical resistance fluctuation (e.g., a transendothelial electrical resistance (TEER) is incorporated into the device in order to monitor a degree of cell-cell contact, cell barrier function, and/or other tissue functions. Changes in resistance measurements have an inverse relationship with tissue permeability and tissue confluence on a layer, providing a quantitative method for rapid analysis of cell-cell contact, cell barrier function, and/or other tissue functions.

### Drug Screening Methods

The present disclosure provides drug screening methods for identifying a candidate agent that modulates a characteristic of a plurality of cells. The methods generally involve: a) introducing a plurality of cells into the cell culture channel of a cell culture device of the present disclosure; b) introducing a cell culture medium into the media channels of the device; c) contacting the cells with the candidate agent; d) maintaining the device under suitable cell culture conditions; and e) measuring a characteristic of the cells using the sensor. A change in the characteristic of the cells in the presence of the candidate agent compared to a characteristic of the cells in the absence of the candidate agent indicates that the candidate agent has use in modulating the characteristic of the cells. Such methods are useful for, e.g., identifying a candidate agent for treating a cardiac condition or disease.

In some cases, the cells used in a subject drug screening method may comprise cardiomyocytes, where cardiomyocytes can be any of the cardiomyocytes as described hereinabove. For example, in some cases, the cardiomyocytes exhibit one or more characteristics of a cardiac disease or condition (a cardiac abnormality). For example in some cases, the cardiomyocytes exhibit one or more characteristics of ischemic heart disease, arrhythmia, tachycardia, bradycardia, myocardial infarction, or a congenital heart condition.

In some cases, the cells used in a subject drug screening method comprise stem cells. In some cases, the cells used in a subject drug screening method comprise induced pluripotent stem cells. In some cases, the cells used in a subject drug screening method are human cells, e.g., human cardiomyocytes, human cardiomyocyte precursors (progenitors), or human iPS cells. In some embodiments, the cells used in a subject drug screening method comprise hepatocytes. In some embodiments, the cells used in a subject drug screening method comprise adipocytes.

In some cases, the sensor in a device used in a method of the present disclosure comprises a mechanosensing pillar, and the step of measuring a characteristic of the cells comprises measuring a beat rate and/or a rhythm of the cells by measuring a deflection of the mechanosensing pillar.

In some cases, the sensor in a device used in a method of the present disclosure comprises an electrode, and the step of measuring a characteristic of the cells comprises measuring a beat rate and/or a rhythm of the cells by measuring a voltage potential of the electrode.

In some instances, a method of the present disclosure for identifying a candidate agent that modulates a characteristic of a plurality of cells comprises: a) introducing a plurality of stem cells into the cell culture channel of a cell culture device of the present disclosure; b) differentiating the cells into a lineage; c) introducing a cell culture medium into the media channels of the device; d) contacting the cells with the candidate agent; e) maintaining the device under suitable cell culture conditions; and f) measuring a characteristic of the cells using the sensor.

In some cases, the cells used in a subject drug screening method are genetically modified cells. In some cases, the method involves genetically modifying the cells after the cells have been introduced into the cell culture channel of the cell culture device.

In some instances, a method of the present disclosure for identifying a candidate agent that modulates a characteristic of a plurality of cells further comprises blocking at least one of the media channels of the device to simulate a disease state by reducing an amount of a nutrient and/or an amount of oxygen that is delivered to the cells from the media channel. For example, in some cases, a method of the present disclosure for identifying a candidate agent that modulates a characteristic of a plurality of cells comprises: a) introducing a plurality of cells into the cell culture channel of a cell culture device of the present disclosure; b) introducing a cell culture medium into the media channels of the device; c) blocking at least one of the media channels of the device to simulate a disease state by reducing an amount of a nutrient and/or an amount of oxygen that is delivered to the cells from the media channel; d) contacting the cells with the candidate agent; e) maintaining the device under suitable cell culture conditions; and f) measuring a characteristic of the cells using the sensor.

In some instances, a method of the present disclosure for identifying a candidate agent that modulates a characteristic of a plurality of cells further comprises modulating a dimension of the device to simulate a disease state by reducing an amount of a nutrient and/or an amount of oxygen that is delivered to the cells from the media channel. For example, in some cases, a method of the present disclosure for identifying a candidate agent that modulates a characteristic of a plurality of cells comprises: a) introducing a plurality of cells into the cell culture channel of a cell culture device of the present disclosure; b) introducing a cell culture medium into the media channels of the device; c) modulating a dimension of the device to simulate a disease state by reducing an amount of a nutrient and/or an amount of oxygen that is delivered to the cells from the media channel; d) contacting the cells with the candidate agent; e) maintaining the device under suitable cell culture conditions; and f) measuring a characteristic of the cells using the sensor. For example, in some cases, the dimension of the device that is modulated is the width of the cell culture channel.

As discussed above, in some cases, a plurality of cardiomyocytes that are cultured in the subject devices exhibit one or more characteristics of a cardiac disease or condition. For example, in some cases, the cardiomyocytes are obtained from an individual having a cardiac disease or condition, or are generated from somatic cells from an individual having a cardiac disease or condition, or are generated from iPS cells generated from somatic cells from an individual having a cardiac disease or condition. In some cases, the cardiomyocytes are genetically modified such that the genetically modified cardiomyocyte exhibits one or more characteristics of a cardiac disease or condition. In some cases, isogenic cardiomyocytes, as described above, are used.

Drugs or test agents may be individual small molecules of choice (e.g., a lead compound from a previous drug screen) or in some cases, the drugs or test agents to be screened come from a combinatorial library, e.g., a collection of diverse chemical compounds generated by either chemical synthesis or biological synthesis by combining a number of chemical “building blocks.” For example, a linear combinatorial chemical library such as a polypeptide library is formed by combining a set of amino acids in every possible way for a given compound length (e.g., the number of amino acids in a polypeptide compound). Millions of test agents (e.g., chemical compounds) can be synthesized through such combinatorial mixing of chemical building blocks. Indeed, theoretically, the systematic, combinatorial mixing of 100 interchangeable chemical building blocks results in the synthesis of 100 million tetrameric compounds or 10 billion pentameric compounds. See, e.g., Gallop et al. (1994), J. Med. Chem 37(9), 1233. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

In some embodiments, a cell (e.g., a cardiomyocyte or cardiac progenitor, a hepatocyte, an adipocyte) is contacted with a test agent in a subject device, as described above, and the effect, if any, of the test agent on a biological activity of the cell is assessed, where a test agent that has an effect on a biological activity of the cell is a candidate agent for treating a disorder or condition. For example, a test agent of interest is one that increases a biological activity of a cardiomyocyte or cardiac progenitor by at least about 5%, at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the biological activity in the absence of the test agent. A test agent of interest is a candidate agent for treating a disorder or condition.

A “biological activity” includes, e.g., one or more of marker expression (e.g., cardiomyocyte-specific marker expression), receptor binding, ion channel activity, contractile activity, and electrophysiological activity.

For example, in some embodiments, the effect, if any, of the test agent on expression of a cardiomyocyte marker is assessed. Cardiomyocyte markers include, e.g., cardiac troponin I (cTnI), cardiac troponin T (cTnT), sarcomeric myosin heavy chain (MHC), GATA-4, Nkx2.5, N-cadherin, β-adrenoceptor (β1-AR), a member of the MEF-2 family of transcription factors, creatine kinase MB (CK-MB), myoglobin, and atrial natriuretic factor (ANF).

As another example, the effect, if any, of the test agent on electrophysiology of a cardiomyocyte or cardiac progenitor is assessed.

As another example, in some embodiments, the effect, if any, of the test agent on ligand-gated ion channel activity is assessed. As another example, in some embodiments, the effect, if any, of the test agent on voltage-gated ion channel activity is assessed. The effect of a test agent on ion channel activity is readily assessed using standard assays, e.g., by measuring the level of an intracellular ion (e.g., Na⁺, Ca²⁺, K⁺, etc.). A change in the intracellular concentration of an ion can be detected using an indicator (e.g., a chemical indicator; a genetically encoded indicator) appropriate to the ion whose influx is controlled by the channel. For example, where the ion channel is a potassium ion channel, a potassium-detecting dye is used; where the ion channel is a calcium ion channel, a calcium-detecting dye is used; etc. As noted above, a genetically encoded calcium indicator can be used.

Suitable intracellular K⁺ ion-detecting dyes include, but are not limited to, K⁺-binding benzofuran isophthalate and the like.

Suitable intracellular Ca²⁺ ion-detecting dyes include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes, and others (see, e.g., the catalog or Internet site for Molecular Probes, Eugene, see, also, Nuccitelli, ed., Methods in Cell Biology, Volume 40: A Practical Guide to the Study of Calcium in Living Cells, Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis, Second Ed, Academic Press (1999); Calcium Signaling Protocols (Methods in Molecular Biology), 2005, D. G. Lamber, ed., Humana Press.)

In some embodiments, screening of test agents is conducted using cardiomyocytes or cardiac progenitors that display an abnormal cellular phenotype (e.g., abnormal cell morphology, gene expression, or signaling), associated with a health condition or a predisposition to the health condition (e.g., a cardiac condition). Such assays may include contacting a test population of cardiomyocytes or cardiac progenitors (e.g., generated from one or more iPS donors exhibiting a cardiac disease or condition) with a test compound; and contacting with a negative control compound a negative control population of cardiomyocytes or cardiac progenitors (e.g., generated from one or more iPS donors exhibiting the cardiac disease or condition). The assayed cellular phenotype associated with the cardiac disease or condition of interest in the test and negative control populations can then be compared to a normal cellular phenotype. Where the assayed cellular phenotype in the test population is determined as being closer to a normal cellular phenotype than that exhibited by the negative control population, the drug candidate compound is identified as normalizing the phenotype.

The effect of a test agent in the assays described herein can be assessed using any standard assay to observe phenotype or activity of a cell (e.g., a cardiomyocyte or cardiac progenitor), such as marker expression, receptor binding, contractile activity, or electrophysiology. For example, in some cases, pharmaceutical candidates are tested for their effect on contractile activity, such as whether they increase or decrease the extent or frequency of contraction. Where an effect is observed, the concentration of the compound can be titrated to determine the half-maximal effective dose (ED50).

### Test Agent/Drug Toxicity

A method of the present disclosure can be used to assess the toxicity of a test agent, or drug, e.g., a test agent or drug designed to have a pharmacological effect on a cell (e.g., a cardiac progenitor or cardiomyocyte), e.g., a test agent or drug designed to have effects on cells other than cardiac progenitors or cardiomyocytes but potentially affecting cardiac progenitors or cardiomyocytes as an unintended consequence. In some embodiments, the disclosure provides methods for evaluating the toxic effects of a drug, test agent, or other factor, in a human or non-human (e.g., murine; lagomorph; non-human primate) subject, comprising contacting one or more cells with a dose of a drug, test agent, or other factor and assaying the contacted cells for markers of toxicity or cardiotoxicity, e.g., for effects of the drug on mechanical properties, such as contractility, of a plurality of cardiomyocytes; or for effects of the drug on electrical properties of a plurality of cardiomyocytes.

Any method known in the art may be used to evaluate the toxicity or adverse effects of a test agent or drug on a cell (e.g., on cardiomyocytes or cardiac progenitors). Cytotoxicity or cardiotoxicity can be determined, e.g., by the effect on cell viability, survival, morphology, and the expression of certain markers and receptors. For example, biochemical markers of myocardial cell necrosis (e.g., cardiac troponin T and I (cTnT, cTnI)) may be used to assess drug-induced toxicity or adverse reactions in cardiomyocytes or cardiac progenitors, where the presence of such markers in extracellular fluid (e.g., cell culture medium) can indicate necrosis. See, e.g., Gaze and Collinson (2005) Expert Opin Drug Metab Toxicol 1(4):715-725. In another example, lactate dehydrogenase is used to assess drug-induced toxicity or adverse reactions in cardiomyocytes or cardiac progenitors. See, e.g., Inoue et al. (2007) AATEX 14, Special Issue: 457-462. In another example, the effects of a drug on chromosomal DNA can be determined by measuring DNA synthesis or repair and used to assess drug-induced toxicity or adverse reactions in cardiomyocytes or cardiac progenitors. In still another example, the rate, degree, and/or timing of [³H]-thymidine or BrdU incorporation may be evaluated to assess drug-induced toxicity or adverse reactions in cardiomyocytes or cardiac progenitors. In yet another example, evaluating the rate or nature of sister chromatid exchange, determined by metaphase spread, can be used to assess drug-induced toxicity or adverse reactions in cardiomyocytes or cardiac progenitors. See, e.g., A. Vickers (pp 375-410 in In vitro Methods in Pharmaceutical Research, Academic Press, 1997). In yet another example, assays to measure electrophysiology or activity of ion-gated channels (e.g., Calcium-gated channels) can be used to assess drug-induced toxicity or adverse reactions in cardiomyocytes or cardiac progenitors. In still another example, contractile activity (e.g., frequency of contraction) can be used to assess drug-induced toxicity or adverse reactions in cardiomyocytes or cardiac progenitors.

Thus, the present disclosure provides a method of evaluating an effect of an agent on a plurality of cells, the method comprising: a) introducing a plurality of cells into the cell culture channel of a cell culture device of the present disclosure; b) introducing a cell culture medium into the media channels of the device; c) contacting the cells with the agent; d) maintaining the device under suitable cell culture conditions; and e) measuring a characteristic of the cells using the sensor. A change in the characteristic of the cells in the presence of the agent compared to a characteristic of the cells in the absence of the agent indicates that the agent modulates the characteristic of the cells. Characteristics include mechanical characteristics, such as contractility; and electrical characteristics such as voltage potential across a cell membrane.

In some cases, the cells used in a subject method of evaluating an effect of an agent on a plurality of cells comprise cardiomyocytes, where cardiomyocytes can be any of the cardiomyocytes as described hereinabove. For example, in some cases, the cardiomyocytes exhibit one or more characteristics of a cardiac disease or condition (a cardiac abnormality). For example in some cases, the cardiomyocytes exhibit one or more characteristics of ischemic heart disease, arrhythmia, tachycardia, bradycardia, myocardial infarction, or a congenital heart condition.

In some cases, the cells used in a subject method of evaluating an effect of an agent on a plurality of cells comprise stem cells. In some cases, the cells used in a subject method of evaluating an effect of an agent on a plurality of cells comprise induced pluripotent stem cells. In some cases, the cells used in a subject method of evaluating an effect of an agent on a plurality of cells are human cells, e.g., human cardiomyocytes, human cardiomyocyte precursors (progenitors), or human iPS cells.

In some cases, the sensor in the device used in a subject method of evaluating an effect of an agent on a plurality of cells comprises a mechanosensing pillar, and the evaluating step comprises measuring a characteristic of the cells comprises measuring a beat rate and/or a rhythm of the cells by measuring a deflection of the mechanosensing pillar.

In some cases, the sensor in the device used in a subject method of evaluating an effect of an agent on a plurality of cells comprises an electrode, and the evaluating step comprises measuring a characteristic of the cells comprises measuring a beat rate and/or a rhythm of the cells by measuring a voltage potential of the electrode.

In some cases, the method comprises differentiating the cells (e.g., stem cells, such as iPS cells) into a lineage, e.g., a cardiomyocyte lineage. Stems cells (e.g., iPS cells) can be induced to become cardiomyocytes before being introduced into (loaded into) a device of the present disclosure. Stems cells (e.g., iPS cells) can be induced to become cardiomyocytes when the stem cells (e.g., iPS cells) are already loaded in a device of the present disclosure.

In some cases, the method further comprises genetically modifying the cells.

In some cases, the method further comprises blocking at least one of the media channels of the device to simulate a disease state by reducing an amount of a nutrient and/or an amount of oxygen that is delivered to the cells from the media channel.

In some cases, the method further comprises modulating a dimension of the device to simulate a disease state by reducing an amount of a nutrient and/or an amount of oxygen that is delivered to the cells from the media channel. In some instances, the device that is modulated is the width of the cell culture channel.

In some embodiments, the present disclosure provides methods for reducing the risk of drug toxicity in a human or murine subject, comprising contacting one or more cardiomyocytes or cardiac progenitors with a dose of a drug, test agent, or pharmacological agent, assaying the contacted one or more differentiated cells for toxicity, and prescribing or administering the pharmacological agent to the subject if the assay is negative for toxicity in the contacted cells. In some embodiments, the present disclosure provides methods for reducing the risk of drug toxicity in a human or murine subject, comprising contacting one or more cardiomyocytes or cardiac progenitors with a dose of a pharmacological agent, assaying the contacted one or more differentiated cells for toxicity, and prescribing or administering the pharmacological agent to the subject if the assay indicates a low risk or no risk for toxicity in the contacted cells.

### Predicting Patient Response

The present disclosure provides methods for predicting patient response to a drug, the method generally involving a) introducing a plurality of cells (e.g., cardiomyocytes; cardiomyocyte progenitors; iPS cells, hepatocytes, adipocytes) into the cell culture channel of a cell culture device of the present disclosure; b) introducing a cell culture medium into the media channels of the device; c) contacting the cells with the drug; d) maintaining the device under suitable cell culture conditions; and e) measuring a characteristic of the cells using the sensor. A change in the characteristic of the cells in the presence of the drug compared to a characteristic of the cells in the absence of the drug indicates that the drug modulates the characteristic of the cells. In some cases, the method further comprises preparing a report indicating that: i) the drug exhibited an undesirable effect on one or more cell characteristics; ii) the drug exhibited no detectable undesirable effects on one or more cell characteristics; or iii) further evaluation of the drug is required. In some cases, e.g., where the report indicates that the drug exhibited an undesirable effect on one or more cell characteristics, the method could further include preparing a report recommending that: i) use of the drug be discontinued in the patient from whom the cells were obtained and to whom the drug has been administered; or ii) the drug not be administered to the patient from whom the cells were obtained.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Cell Culture, Differentiation and Multi-Organ Circulation to Evaluate a Candidate Agent

Multi-organ circulation is modeled using a master chip that comprises a plurality of microfluidic cell culture units, and a plurality of connectors that connect the microfluidic cell culture units to each other. First, stem cells are loaded into each of the microfluidic cell culture units. Next, a cell culture medium comprising a differentiation factor is introduced into each of the individual microfluidic cell culture units to differentiate the stem cells into a target tissue type. Next, the cell culture units with the differentiated and matured cells are are fed with a universal cell culture medium and are then connected by plugging suitable connectors into the master chip. The connectors are pre-filled with a universal cell culture medium. A volumetric flow rate between each of the microfluidic cell culture units is specified using appropriate connector geometries and dimensions. The universal cell culture medium is then moved between the individual microfluidic cell culture units at the designated volumetric flow rate(s). A candidate agent is then introduced into the system, and the effect of the candidate agent on each of the individual cell types is monitored using in situ and ex situ measurement techniques.

### Example 2: Combined Culture of Heart, Lung and Liver Tissue with Uni-Directional Circulation

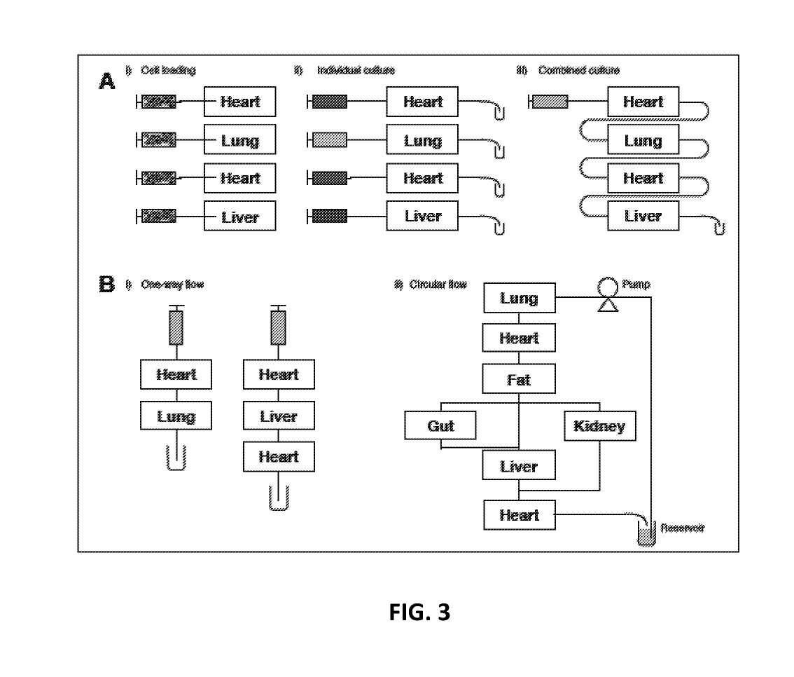

Stem cells are loaded into four different microfluidic cell culture units on a master chip and fed with stem cell culture medium. Cell differentiation protocols are conducted to differentiate the cells into the cell types depicted in FIG. 3, panel A. A specific cell differentiation factor is injected into each microfluidic cell culture unit to differentiate the stem cells therein into a target tissue type. After successful differentiation into cardiomyocytes and hepatocytes, the tissues are individually fed with a specific cell culture medium until a desired tissue maturity is reached. Next, all of the microfluidic cell culture units are switched to a universal cell culture medium. Next, the microfluidic cell culture units are connected in series as depicted in FIG. 3, panel A, iii). The universal cell culture medium is introduced into the first microfluidic cell culture unit (containing cardiomyocytes) via an infusion pump and is moved through each of the microfluidic cell culture units via the connectors at a specified volumetric flow rate. After passing through the microfluidic cell culture unit containing hepatocytes, the cell culture medium is collected in a receptacle.

Once the circulation pattern has been established, a pharmaceutical compound is introduced into the cell culture medium and the effect of the pharmaceutical compound on the cells is measured. For instance, the effect of the pharmaceutical compound on the beat rate and electrophysiology of the cardiomyocytes is measured, and the metabolic functionality of the hepatocytes is characterized to evaluate the impact of the pharmaceutical compound on the cells.

### Example 3: Combined Culture of Lung, Heart, Fat, Gut, Kidney and Liver Tissue with Continuous Circulation

Specific cell types as depicted in FIG. 3, panel B, ii) are loaded into the microfluidic cell culture units of a master chip. The tissues are individually fed with a specific cell culture medium until a desired tissue maturity is reached. Next, all of the microfluidic cell culture units are switched to a universal cell culture medium. Next, the microfluidic cell culture units are connected, either in series or in parallel, as depicted in FIG. 3, panel B, ii). The universal cell culture medium is introduced into the first microfluidic cell culture unit (containing lung cells) via an infusion pump and is moved through each of the microfluidic cell culture units via the connectors at a specified volumetric flow rate. After passing through the microfluidic cell culture unit containing heart cells (cardiomyocytes), the cell culture medium is collected in a reservoir and is then recirculated to the first microfluidic cell culture unit containing lung cells. The recirculation of the universal cell culture medium through the system mimics continuous circulation in a subject.

Once the circulation pattern has been established, a pharmaceutical compound is introduced into the cell culture medium and the effect of the pharmaceutical compound on the cells is measured. For instance, the effect of the pharmaceutical compound on the beat rate and electrophysiology of the cardiomyocytes is measured, and the metabolic functionality of the hepatocytes is characterized to evaluate the impact of the pharmaceutical compound on the cells.

### Example 4: Generation and Characterization of a Multi-Organ μOrgano System

This example describes generation and characterization of a multi-organ μOrgano system. The μOrgano system is a microphysiological system (MPS). MPS are also referred to in the art as “organ-on-a-chip” systems. The present multi-organ μOrgano system is customizable, and enables fluidic control of μL volumes. The present multi-organ μOrgano system is specifically designed to connect multiple organ-on-a-chip (μ-organs) systems into multi-organ-chips. The present μOrgano system is a plug & play system that allows for: i) separate loading of different cell types; ii) temporal control of individual culture of cells for differentiation and tissue development; and, iii) subsequent temporal control of fluidic connections of the individual tissues, as depicted in FIG. 4A-4B.

### Materials and Methods

### Fabrication of Connectors

To create the connectors, 45 μm high and wide square channel structures were patterned with SU8 3050 photoresist (MicroChem Corp, Newton, Mass.) onto silicon wafers (University Wafer, Boston, Mass.) according to the manufacturer's data sheets. Subsequently, 1.5 mm high posts (diameter 2 mm) were patterned at the designated locations for in- and outlet ports. Six layers of 250 μm thick SU8 100 photoresist (MicroChem Corp, Westborough, Mass.) were spin-coated (10 s at 500 rpm+30 s at 1000 rpm) on top of the channel structures. Following each individual spin-coating step, the wafers were baked for 15 min at 65° C. and 2 hours at 95° C. The entire coating process was then finalized by a soft bake at 95° C. for 12 hours. The patterning was achieved by exposing the coated wafers to 33 mW/cm² UV light using a mask aligner (Hybralign Series 200, OAI, San Jose, Calif.) for a total of 4 min (60 s exposures interrupted by 2 min cool down times). The exposed wafers were then developed, baked for 24 hours at 40° C., and functionalized using a Tridecafluoro-1,1,2,2-Tetrahydrooctyl)Trichlorosilane (Gelest, Morrisville, Pa.). By performing exclusion molding on these wafers, connectors with prefabricated in- and outlet holes were fabricated. Briefly, uncured polydimethylsiloxane (PDMS, Sylgard 184, Down Corning, Midland, Mich.)—1:10 w/w ratio of curing agent to prepolymer—was poured onto the wafer and subsequently covered with a mylar sheet, which was clamped onto the wafer using a glass slide. After overnight curing at 60° C., the mold was peeled from both the wafer and the mylar sheet. The molded connectors were then cut into individual modules, which were then bonded to microscope glass slides by exposing them to oxygen plasma (Plasma Equipment Technical Services, Livermore, Calif.) at 60 W for 20 s and subsequent baking at 60° C. for 3 h. Glass capillaries (Micro Bore Tubings, Accu-Glass, St. Louis, Mo.) were manually cut using a capillary cutting stone (Hampton Research, Aliso Viejo, Calif.) and subsequently boiled in Milli-Q water for 1 h in order to dull the edges and prevent damaging the PDMS devices. Following a cleaning step using 1 M sodium hydroxide for 1 h at room temperature, the capillaries were bonded into the in- and outlet ports of the connectors by exposing them to oxygen plasma at 60 W for 20 s and subsequent baking at 60° C. for 3 h.

### Fabrication of MPSs

The cardiac MPSs were fabricated via a two-step photolithography process as described in Mathur et al. (2015) Sci Rep 5:8883-3. Briefly, in the first step, 2 μm high “endothelial-like” barriers and a weir gap were patterned via UV lithography using SU-8 2001 photoresist (MicroChem Corp) a first step. In the second step, the 35 μm high media and cell culture channels were fabricated using SU-8 3025 (MicroChem Corp). The patterned wafers were then baked, and coated with trichloro-1H, 1H, 2H, 2H-perfluorooctylsilane (FOTS, Gelest, Pa., USA). MPSs were replica molded by pouring uncured PDMS (Sylgard 184, Down Corning, Midland, Mich.)—1:10 w/w ratio of curing agent to prepolymer—onto the master wafer and cured overnight at 60° C. PDMS devices were aligned and bonded to microscope glass slides after exposure to oxygen plasma (Plasma Equipment Technical Services, Livermore, Calif.) at 60 W for 20 s. To stabilize bonding, the devices were subsequently baked at 60° C. for 3 h.

### Connection of MPSs

Before usage, the sterility of the connectors had to be ensured. Therefore, the modules were flushed for 30 min with 70% ethanol, subsequently washed with PBS for 30 min using a PhD Ultra syringe pump, and stored under sterile conditions Immediately prior to the experiments, the connectors were prefilled with the respective cell culture medium. After carefully removing the tubings necessary for the separate feeding from the out- and inlet ports of the MPSs, the prefilled connectors were inserted into the respective ports under sterile conditions. The connected systems were then fed using continuous flow from a syringe pump and placed under standard cell culture conditions. To validate the bubble-free connection, the connectors were prefilled with food dye (DecACake) coloured Milli-Q water and a bright field microscope was focused on a section of the media channel in the immediate proximity of the inlet of the (defined by the media flow) second device. Video microscopy data taken at the temporal onset of the media flow showed the replacement of the colourless by the coloured liquid and was then analysed for the occurrence of bubbles. To characterize the transport times necessary for the media to flow from one cell chamber to the next one, systems consisting of two MPSs connected via linear connectors featuring capillaries of different inner diameters (IDs) were prefabricated. These systems were infused with food dye coloured Milli-Q water at a rate of 20 μL per hour via syringe pump and the time manually measured via visualization of the flow under a microscope. To test the reproducibility of the bifurcation, multiple systems consisting of three MPSs each were connected with bifurcation connectors. Dye coloured Milli-Q water was pumped at a rate of 20 tit per hour via syringe pump into the first MPS, the media was collected from the outlet ports of the two other MPSs in neighboring reservoirs (placed in a closed humid environment next to sacrificial water containers), and the volumes in both of these reservoirs were determined after 20 h.

### Loading of MPSs and Tissue Characterization

Cardiac tissues inside the MPSs were generated as described in Mathur et al., Sci. Rep., 2015. 5:8883-3. Briefly, human CMs were derived from hiPSCs via modulation of the WNT pathway, using an optimized directed cardiac differentiation protocol. Mathur et al., Sci. Rep., 2015. 5:8883-3; Lian et al. (2012) Proc Natl Acad Sci 109:E1848-57. At day 15 of the differentiation process, the beating CMs were dissociated using a singularization protocol introduced by Zhu et al. (2011) Methods Mol Biol 767:419-31 The cell chambers of the MPSs were pre-coated with fibronectin (20 μg/mL in PBS) for 1 h at 37° C. subsequent to hydrophilizing and sterilizing them for 3 minutes at 180 W using 02 plasma (PETS Reactive Ion Etcher). Cells were loaded into the MPS by applying 100-200 μL of a cell solution (4-5 million cells/mL) to the cell inlet port and employing a negative pressure at the outlet ports utilizing a PhD Ultra syringe pump (Harvard Apparatus). The loaded devices were then fed using a syringe pump with a continuous flow of EB20 media (Knockout DMEM supplemented with 20% fetal bovine serum (FBS), 2 mM L-glutamine, 1×MEM non-essential amino acids (MEM-NEAA), 400 nM 2-mercaptoethanol) (Life Technologies). For the first 24 h the media was supplemented with 10 μM Y-27632 (BioVision). After successful formation of a robust tissue with homogeneous beating behavior, the feeding was continued with serum-free media (RPMI 1640 containing B27 with insulin supplement). The loading of 3T3 fibroblasts into the MPSs was performed analogously with the only difference being the feeding media, which consisted of DMEM (Invitrogen) supplemented with 10% FBS and 1% Pen/Strep. To characterize viability, the connected MPSs were washed with sterile PBS (Corning) via syringe pump infusion at a rate of 5 μL/min for 15 minutes. Following this, cells were stained using a solution of 2 μM Calcein®, AM and 4 μM ethidium homodimer-1 (Life Technologies) in sterile PBS, infused via pump at 5 μL/min for 45 minutes. After staining, devices were imaged via fluorescent microscopy (Nikon Eclipse TE300, Nikon, Tokyo, Japan). To characterize functionality of the cardiac tissues, bright field movies of the beating tissues inside the MPSs were taken using the Nikon Eclipse TE300 microscope fitted with a “QICAM Fast” camera (Qlmaging, Surrey, BC, Canada). These movies were subsequently analysed using our custom motion tracking software (available under a GNU license at http://gladstone.ucsf.edu/46749d811/; Matlab-based (MathWorks, Natick, Mass.)) utilizing parallel computing on all cores of a 12-core Mac Pro (Apple, Cupertino, Calif.) as described in Huebsch (2015) Tissue Eng Part C 21:467-79. The block matching based software quantifies the beating motion and outputs motion kinetics with characteristic beating and relaxation peaks allowing for quantification of parameters such as beat rate.

### Results

The basic building blocks of the μOrgano were: i) a master-organ-chip; and, ii) plug & play connectors. The master-organ-chip consisted of a grid-like arrangement of individual MPSs (FIG. 4B). These MPSs can be a custom combination of different organ-on-a-chip systems including, but not limited to the systems described in, e.g., Mathur et al., Sci. Rep., 2015. 5:8883-3; Huh et al., Science, 2010. 328:1662-8; Lee et al., Biotechnol. Bioeng., 2007. 97:1340-6; Jang and Suh, Lap Chip, 2010. 10:36-42; and Hsu et al., Lap Chip, 2013. 13:2990. The sole prerequisite for a compatible MPS was that it contains defined media inlet and outlet ports, which can be arranged on an equidistant grid. Other than this prerequisite, there were no limitations in terms of design and characteristics of the MPSs. As a proof of concept, master-organ-chips consisting of multiple units of the cardiac MPS recently introduced by Mathur et al. (Sci. Rep., 2015. 5:8883-3) were focused on. This MPS consisted of a central cell chamber, two adjacent media channels, and arrays of connecting microchannels. This design created purely diffusive transport of media compounds between the media channels and the cell chamber, with diffusion properties similar to the endothelial barrier present in the human in vivo vasculature.

The plug & play connectors consisted of small microfluidic devices featuring channel structures, and inlets and outlets equipped with open cylinders (FIG. 4B). The length of the cylinder corresponded to the combined thickness of master-organ-chip and connectors. These connectors can be “plugged” into the in- and outlet ports of the master-organ-chip and thereby used to connect two (or more) individual MPS units (FIG. 4B). The channel structures ranged from simple linear channels (length matching n×grid constant or n×√2×grid constant) connecting two neighbouring MPS units to more complex structures such as bifurcations (FIG. 4B), which split the flow to two different MPS units. Bifurcations with different channel widths allowed for a controlled distribution of dissimilar flows into different MPSs. The combination of multiple bifurcations and/or linear connectors enabled complex systems providing a further step towards the recapitulation of the in vivo circulation. In general, a toolbox of connectors with various structures enabled the creation of customized circulation architectures.

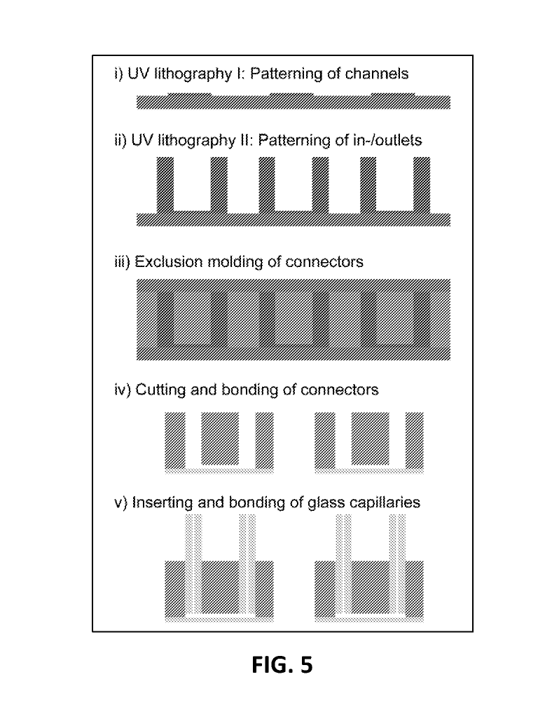

The fabrication and practical implementation of the tiOrgano system provided three major challenges: i) precise and reproducible in- and outlet positions were necessary to allow for a plug & play connection; ii) the dead volume inside the connectors needed to be minimized in order to have physiological transport times for the media to travel from one MPS to the next one; and, iii) the insertion of the plug & play connectors must result in a sealed and bubble free system. To create in- and outlet ports in the connectors with precise spatial orientation and reproducible straight vertical channels, manual punching using biopsy punches commonly used for fabrication of microfluidic devices was not feasible. Previous attempts to directly fabricate ports placed posts manually on the master before the replica molding (Duffy et al., Anal. Chem., 1998. 70:4974-84) or utilized double casting approaches with either manual coring of the intermediate molds (Desai et al., Lap Chip, 2009. 9:1631) or a combination of milling and hot-embossing (Park and Han, Biomed. Microdevices, 2010. 12:345-51). Any type of manual handling as well as double casting was avoided and it was thereby achieved a higher precision and spatial resolution by employing a combination of multi-step UV lithography and exclusion molding of the PDMS devices as depicted in FIG. 5: First, microscale channel structures of appropriate geometries for the different types of connectors (Linear, bifurcation, . . . ) were patterned. Subsequently, macroscale posts as templates for the in- and outlet ports were patterned precisely at the respective ends of the channels.

To minimize the dead volume inside the connectors, it was essential to reduce the volume of the tubes in the in- and outlet ports, since these were the sole components not created by microfabrication. This requirement is not sufficiently met by stainless steel catheter couplers (typically 1.3-3 μL/10 mm tube) commonly utilized for microfluidic devices. A useful alternative is the use of glass capillaries (Micro Bore Tubings, Accu-Glass, St. Louis, Mo.) with small IDs of 50˜200 μm (≈0.02˜0.3 μL/10 mm tube). These capillaries were bonded in the connector ports resulting in Lego®-type connector modules with small volumes. The resulting volume of the connectors was defined by the volume of the connecting channel (≈0.026 μl for in-series modules), the volume of the capillaries (≈0.016 μL for capillaries (8 mm) with 50 μm ID; ≈0.2 μL in case of 150 μm ID), and the volumes between the end of the capillaries and the glass slide. To obtain the entire “inter-MPS volume”, the channel volume of two halves of the MPS (≈0.108 μL total for the cardiac MPS) had to be taken into account as well. The actual choice of ID for the capillaries required a balancing of minimization of dead volume and hydraulic resistance. Hydraulic resistances can be obtained using

$${R}_{circ} = \frac{8 μ}{π} \frac{l}{{R}^{4}}$$

for channels with circular crossections (length L, radius R) and

$${R}_{rec} = \frac{12 μ l}{( \frac{w}{h} - 0.63 ) {h}^{*}}$$

for channels with rectangular crossection (length L, width w>height h) by assuming a viscosity μ=0.78 mPa s (Dulbecco's modified eagle medium (DMEM) with supplements at 37° C.) Bacabac et al., J. Biomech., 2005. 38:159-67). As detailed in Table 1, feeding two connected MPSs with a typical flow rate of 20 μL/h caused a back pressure of approximately 10 mbar when using capillaries with 50 μm ID and about 6 mbar with 150 μm ID. In the case of larger systems with ten MPSs in series, the back pressure reached up to ≈80 mbar and ≈40 mbar respectively. The resulting values, provided no problems for typically used macroscopic pumps as well as most micropumps (Ashraf et al., Int. J. Mol. Sci., 2011. 12:3648-704; Au et al., Micromachines, 2011. 2:179-220), and also allow for the utilization of gravity feeding (approximately 6-80 cm height difference).

TABLE 1
Hydraulic resistance and back pressure occurring at a typical
feeding rate of 20 μL/h for individual MPSs, linear connectors,
and connected systems.

| | Capillary | Hydraulic | |
|---|---|---|---|
| | ID | resistance | Back pressure |
| | (μm) | (mbar/(μL/h)) | (mbar) (for 20 μL/h) |
| MPS | | 0.075 | 1.5 |
| Linear connector | 50 | 0.363 | 7.3 |
| | 150 | 0.140 | 2.8 |
| 2 connected MPSs | 50 | 0.513 | 10.3 |
| | 150 | 0.290 | 5.8 |
| 5 connected MPSs | 50 | 1.827 | 36.5 |
| | 150 | 0.934 | 18.7 |
| 10 connected MPSs | 50 | 4.017 | 80.3 |
| | 150 | 2.008 | 40.2 |

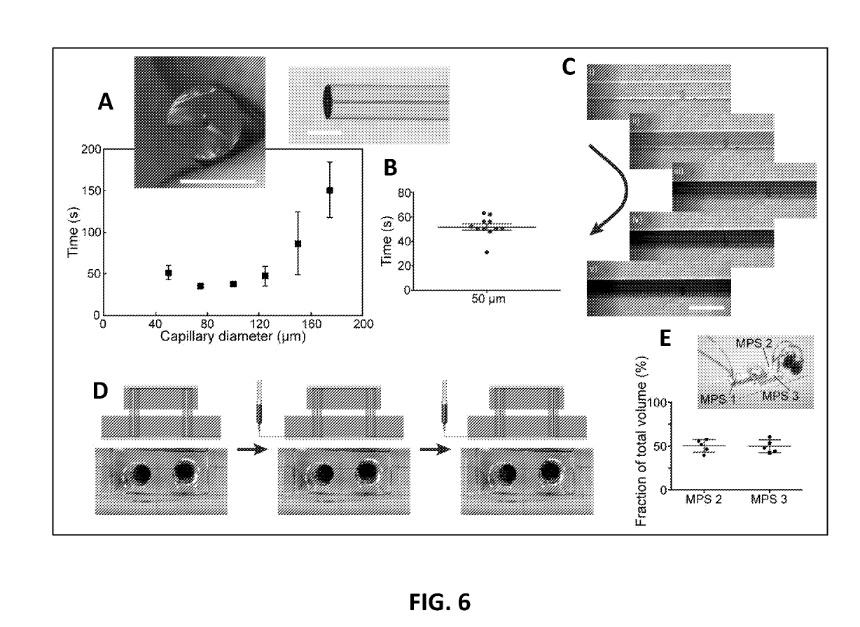

To measure transport times of the media travelling from one MPS to the next one, pairs of cardiac MPSs were connected using a linear connector. By pumping dyed Milli-Q water through the system (20 μL/h) and measuring the time necessary to travel from the cell chamber in MPS 1 to the one in MPS 2, physiological transport times were confirmed in the range of ˜50 s to 150 s for capillaries with various IDs (FIG. 6A), representing transported volumes in the range of ≈0.3-0.8 μL respectively. These experimental values indicated that the previously unknown average spacing between the end of the glass capillaries and the microscope slides were in the range of 10-20 μm. The reproducibility of the connection step was validated by repeating the measurement in ten independent systems, which were connected with connectors featuring capillaries with 50 μm ID, revealing only small variations in the transport times (FIG. 6B). These variations were partly due to slight differences in capillary lengths leading to differences in the inter-MPS volume. Despite these variations the physiological character of transport times was ensured. Additionally, a large-scale automatized fabrication of the capillaries with precise length control would significantly reduce this variability.

A sealed and bubble free system was achieved by both bonding of the capillaries into the connectors, and prefilling of the connectors with the required media before inserting them into the master-organ-chip. Thereby, the media flow after connection takes place without occurrence of air bubbles (FIG. 6C) or leakage (FIG. 6D). To test the performance of the bifurcation connectors in terms of reproducibility and evenness of flow splitting, dye coloured Milli-Q water was pumped into MPSs, which were connected to two MPSs each using bifurcations. Measuring the liquid volumes in the respective outlet ports revealed an even splitting of the input flow (FIG. 6E), whereby the slight variations could again be traced back to small differences in capillary lengths due to the manual cutting process.

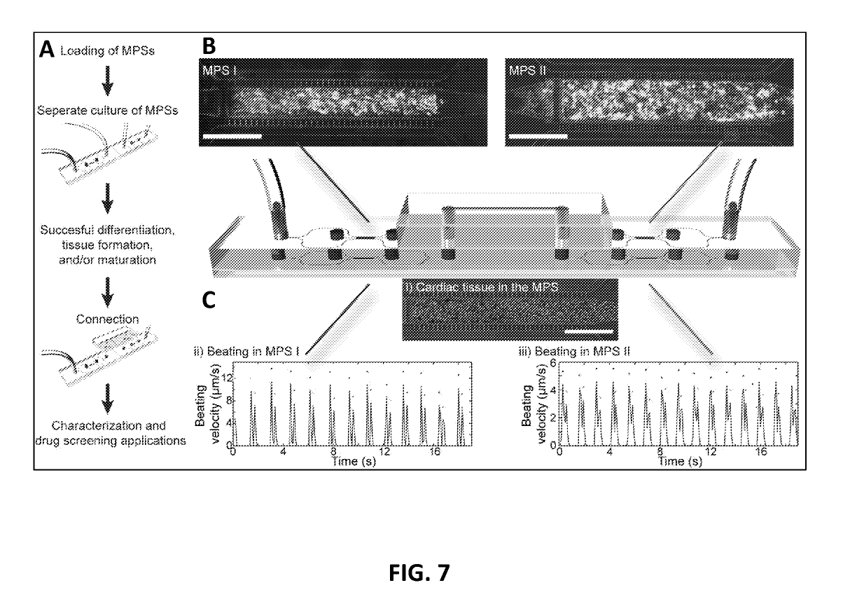

The use of the μOrgano system for cell culture requires sterility of the system in order to prevent contamination. The biocompatible PDMS/glass hybrid modules allowed for standard sterilization methods. As a proof of principle for the applicability for cell culture systems, 3T3 fibroblasts were injected into two MPSs and were cultured separately for 48 h. After connecting them with a linear connector and subsequent in series-culture for another 72 h, a live/dead stain was performed. Fluorescent imaging of the stained MPSs (FIG. 7A) revealed the viability of the cells in both of the connected MPSs, confirming the capability of the μOrgano system to keep cells viable and thereby validating its general applicability for cell culture systems. To validate the ability of the μOrgano system to connect organ-on-a-chip devices while retaining their functionality, functional human cardiac tissue was generated in two MPSs by injecting hiPSC derived cardiomyocytes in two cardiac MPSs Mathur et al. (Sci. Rep., 2015. 5:8883-3). The two devices were then fed independently for 3 days with a serum containing media. To ensure cardiac tissue formation, the media was supplemented with an inhibitor of Rho-associated, coiled-coil containing protein kinase (ROCK) for the first 24 h after loading. After 3 days, robust tissues were formed in each of the two MPSs. The tissues showed homogeneous beating with physiological beat rates. Subsequently, the systems were switched to a serum-free media and connected with a linear μOrgano connector. After 24 h in-series culture, using video microscopy analysis, both devices beat homogeneously at physiological beat rates validating the capability of the μOrgano system to enable the maintenance of a functional phenotype in connected heart-on-a-chip devices (FIG. 7B). Although these heart-on-a-chip devices beat spontaneously at similar rates, the beating was independent from each other, indicating each device behaves as a technical replicate and therefore an array of devices can be used for high content screening during drug development.

FIG. 4: Challenges and solution for multi-organ-systems: FIG. 4A) General requirements for multi-organ-chips: i) initial separate loading of the respective cells; ii) individual culture for differentiation, formation, equilibration, and maturation of the tissues; and, iii) combined culture for drug screening purposes. FIG. 4B) Underlying concept of the μOrgano system: Schematics depicting the basic μOrgano components: the master-organ-chip and exemplary plug & play connectors. Conceptual idea of the usage principle of the μOrgano system for the connection of two MPSs in series via a simple linear channel connector with a close-up of the connected system highlighting the resulting media flow.

FIG. 5: Fabrication of μOrgano building blocks. Schematic protocol for the fabrication of connectors (and MPSs) with precise in- and outlet positions via multi step UV-lithography: i) microscopic channel structures are patterned in photoresist using UV lithography; ii) macroscopic in- and outlets are patterned as pillars on top of the microscopic channel structures using a second UV lithography step; iii) microfluidic PDMS devices are fabricated with predefined in- and outlets via exclusion molding; iv) PDMS connectors are cut and bonded to pre-cut microscope slides; and, v) glass capillaries are inserted and bonded into the in- and outlets of the connectors.

FIG. 6: Characterization of μOrgano building blocks. FIG. 6A) Transition time of the interface of a liquid advancing through a system of two MPSs and a linear connector. The time necessary to advance from the cell chamber in MPS 1 to the cell chamber in MPS 2 is plotted versus the inner diameters of the glass capillaries in the respective systems. Insets show pictures of the respective glass capillaries (scale bars=2 mm). FIG. 6B) Scatter plot of the transition times for ten independent systems connected by the same type of connectors featuring 50 μm ID capillaries. FIG. 6C) Time series of microscopy images from a channel section in the proximity of the inlet of the second MPS initially filled with clear water. The continuous transition occurring after connection to a MPS filled with coloured water using a food dye reveals the bubble less connection ability of the system (scale bar=100 μm). FIG. 6D) Time series of pictures showing two MPSs connected by a linear connector whereby MPS 1 is prefilled with red dyed water, and MPS 2 and the connector with blue dyed water. Pumping red dyed water into MPS 1 leads to the replacement of the blue dyed water in both the connector and MPS 2 without the occurrence of leakage. FIG. 6E) Volume flown through MPS 2 (left; in flow direction) and MPS 3 (right) plotted as percentage of the total volume after connection to MPS 1 via a bifurcation connector.

FIG. 7: Proof of concept of the μOrgano system: FIG. 7A) General procedure for biological experiments with the μOrgano system. FIG. 7B) Combined culture of two devices with 3T3 fibroblasts: Live (green)/dead (red) staining in both devices after 1 day of individual and 2 days of combined culture show that viability can be maintained. FIG. 7C) In-series culture of two heart-on-a-chip devices: tracings of the beating motion of cardiac tissue formed by hiPSC-cardiomyocytes—i) optical microscopy image—in two connected MPSs; ii) MPS 1; iii) MPS 2). The analysis using computational motion tracking reveals that a physiological phenotype is retained and individual cardiac devices beat with distinct frequencies. (scale bars=200 μm)

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A cell culture system comprising:
a plurality of microfluidic cell culture units;
a plurality of connectors that fluidly connect the microfluidic cell culture units to one another; and
a cell culture medium.

2. The system according to claim 1, comprising:
a controller;
a processor; and
a computer-readable medium comprising instructions that, when executed by the processor, cause the controller to move the cell culture medium at a specified volumetric flow rate through a connector between at least two of the microfluidic cell culture units.

3. The system according to claim 1 or claim 2, wherein the number of microfluidic cell culture units ranges from 12 to 100.

4. The system according to any one of claims 1-3, wherein each of the microfluidic cell culture units comprises a cell culture channel and at least one media channel.

5. The system according to claim 4, wherein the media channel comprises an inlet port and an outlet port.

6. The system according to claim 5, wherein the inlet and outlet ports of the media channels are aligned on an equidistant grid.

7. The system according to claim 1, wherein each connector comprises at least one inlet port and at least one outlet port that are connected by one or more channels.

8. The system according to claim 7, wherein at least one connector comprises from 1 to 30 inlet ports.

9. The system according to claim 7, wherein at least one connector comprises from 1 to 30 outlet ports.

10. The system according to claim 7, wherein at least one connector is configured to connect a plurality of microfluidic cell culture units in series.

11. The system according to claim 7, wherein at least one connector is configured to connect a plurality of microfluidic cell culture units in parallel.

12. The system according to claim 7, wherein at least one connector is configured to connect a plurality of microfluidic cell culture units in series and to connect a plurality of microfluidic cell culture units in parallel.

13. The system according to claim 7, wherein at least one connector comprises two or more channels, and wherein the length of each of the channels is the same.

14. The system according to claim 7, wherein at least one connector comprises two or more channels, and wherein the length of one channel is greater than the length of another channel.

15. The system according to claim 7, wherein at least one connector comprises two or more channels, and wherein the cross sectional area of each of the channels is the same.

16. The system according to claim 7, wherein at least one connector comprises two or more channels, and wherein the cross sectional area of one channel is greater than the cross sectional area of another channel.

17. The system according to any one of claims 1-16, wherein at least one of the connectors comprises a sensor that is configured to measure a characteristic of the cell culture medium.

18. The system according to any one of claims 1-17, wherein the cell culture medium is configured to support a plurality of cell types.

19. The system according to any one of claims 1-19, wherein the specified volumetric flow rate is selected from a library of organ-specific parameters.

20. The system according to claim 19, wherein the library of organ-specific parameters includes a fluid constituent consumption rate, a fluid storage rate, and/or a fluid resistance property for a plurality of organs.

21. The system according to any one of claims 1-20, wherein the specified volumetric flow rate through the connector ranges from 10 μL/h to 5 mL/h.

22. A method of culturing cells, the method comprising:
introducing a plurality of cells into the microfluidic cell culture units of the cell culture system according to any one of claims 1-21; and
maintaining the system under suitable cell culture conditions.

23. The method according to claim 22, wherein the cells comprise one or more of: cardiomyocytes; hepatocytes; adipocytes; induced pluripotent stem (iPS) cells; beta islet cells; leukocytes; lung epithelial cells; exocrine secretory epithelial cells; hormone-secreting cells, keratinocytes; lymphocytes; macrophages; monocytes; renal cells; urethral cells; sensory transducer cells; autonomic neuronal cells; central nervous system neurons; glial cells; skeletal muscle cells; osteocytes; osteoblasts; chondrocytes; smooth muscle cells; microglial cells; stromal cells; or progenitor cells thereof.

24. The method according to claim 22, wherein the cells comprise stem cells.

25. The method according to claim 24, wherein the stem cells comprise induced pluripotent stem cells.

26. The method according to any one of claims 22-25, wherein the cells comprise human cells.

27. The method according to any one of claims 22-26, wherein the system comprises a sensor that is adapted to collect data from a plurality of cells and/or a cell culture medium in the system, and wherein the method further comprises collecting data from the sensor.

28. A method for evaluating a plurality of cells in vitro, the method comprising:
introducing a plurality of cells into the microfluidic cell culture units of the cell culture system according to any one of claims 1-21;
maintaining the system under suitable cell culture conditions; and
measuring a characteristic of the cells.

29. The method according to claim 28, wherein the system comprises a sensor that is adapted to collect data from a plurality of cells and/or a cell culture medium in the system, and wherein the method further comprises measuring a characteristic of the cells and/or the cell culture medium using the sensor.

30. The method according to claim 28 or 29, wherein the cells comprise one or more of: cardiomyocytes; hepatocytes; adipocytes; induced pluripotent stem (iPS) cells; beta islet cells; leukocytes; lung epithelial cells; exocrine secretory epithelial cells; hormone-secreting cells, keratinocytes; lymphocytes; macrophages; monocytes; renal cells; urethral cells; sensory transducer cells; autonomic neuronal cells; central nervous system neurons; glial cells; skeletal muscle cells; osteocytes; osteoblasts; chondrocytes; smooth muscle cells; microglial cells; stromal cells; or progenitor cells thereof.

31. The method according to claim 28 or claim 29, wherein the cells comprise stem cells.

32. The method according to claim 31, wherein the stem cells comprise induced pluripotent stem cells.

33. The method according to any one of claims 28-32, wherein the cells comprise human cells.

34. A method for identifying a candidate agent that modulates a characteristic of a plurality of cells, the method comprising:
introducing a plurality of cells into the microfluidic cell culture units of the cell culture system according to any one of claims 1-21;
contacting the cells with the candidate agent;
maintaining the system under suitable cell culture conditions; and
measuring a characteristic of the cells,
wherein a change in the characteristic of the cells in the presence of the candidate agent compared to a characteristic of the cells in the absence of the candidate agent indicates that the candidate agent has use in modulating the characteristic of the cells.

35. The method according to claim 35, wherein the system comprises a sensor that is adapted to collect data from a plurality of cells and/or a cell culture medium in the system, and wherein the method further comprises measuring a characteristic of the cells and/or the cell culture medium using the sensor.

36. The method according to claim 34 or claim 35, wherein the cells comprise one or more of: cardiomyocytes; hepatocytes; adipocytes; induced pluripotent stem (iPS) cells; beta islet cells; leukocytes; lung epithelial cells; exocrine secretory epithelial cells; hormone-secreting cells, keratinocytes; lymphocytes; macrophages; monocytes; endothelial cells; epithelial cells; renal cells; urethral cells; sensory transducer cells; autonomic neuronal cells; central nervous system neurons; glial cells; skeletal muscle cells; osteocytes; osteoblasts; chondrocytes; smooth muscle cells; microglial cells; stromal cells; or progenitor cells thereof.

37. A method for evaluating an effect of an agent on a plurality of cells, the method comprising:
introducing a plurality of cells into the microfluidic cell culture units of the cell culture system according to any one of claims 1-21;
contacting the cells with the agent;
maintaining the system under suitable cell culture conditions; and
measuring a characteristic of the cells,
wherein a change in the characteristic of the cells in the presence of the agent compared to a characteristic of the cells in the absence of the agent indicates that the agent modulates the characteristic of the cells.

38. The method according to claim 37, wherein the device comprises a sensor that is adapted to collect data from a plurality of cells in the system, and wherein the method further comprises measuring a characteristic of the cells using the sensor.

39. The method according to claim 37 or claim 38, wherein the cells comprise one or more of: cardiomyocytes; hepatocytes; adipocytes; induced pluripotent stem (iPS) cells; beta islet cells; leukocytes; lung epithelial cells; exocrine secretory epithelial cells; hormone-secreting cells, keratinocytes; lymphocytes; macrophages; monocytes; renal cells; urethral cells; sensory transducer cells; autonomic neuronal cells; central nervous system neurons; glial cells; skeletal muscle cells; osteocytes; osteoblasts; chondrocytes; smooth muscle cells; microglial cells; stromal cells; or progenitor cells thereof.

40. A method for evaluating an effect of a first plurality of cells on a second plurality of cells, the method comprising:
introducing a plurality of cells into the microfluidic cell culture units of the cell culture system according to claim 1, wherein a first plurality of cells is introduced into a first microfluidic cell culture unit and a second plurality of cells is introduced into a second microfluidic cell culture unit;
maintaining the system under suitable cell culture conditions;
stimulating the first plurality of cells with a stimulus; and
measuring a characteristic of the second plurality of cells,
wherein a change in the characteristic of the second plurality of cells in the presence of the stimulus compared to the characteristic of the second plurality of cells in the absence of the stimulus indicates that stimulating the first plurality of cells modulates a characteristic of the second plurality of cells.

41. The method according to claim 40, wherein the device comprises a sensor that is adapted to collect data from a plurality of cells in the system, and wherein the method further comprises measuring a characteristic of the cells using the sensor.

42. The method according to claim 40 or claim 41, wherein the first plurality of cells comprises a first cell type and the second plurality of cells comprises a second cell type, wherein the first and second cell types are different.

43. The method according to any one of claims 40-42, wherein the first cell type is selected from one or more of: cardiomyocytes; hepatocytes; adipocytes; induced pluripotent stem (iPS) cells; beta islet cells; leukocytes; lung epithelial cells; exocrine secretory epithelial cells; hormone-secreting cells, keratinocytes; lymphocytes; macrophages; monocytes; renal cells; urethral cells; sensory transducer cells; autonomic neuronal cells; central nervous system neurons; glial cells; skeletal muscle cells; osteocytes; osteoblasts; chondrocytes; smooth muscle cells; microglial cells; stromal cells; or progenitor cells thereof, and wherein the second cell type is selected from one or more of: cardiomyocytes; hepatocytes; adipocytes; induced pluripotent stem (iPS) cells; beta islet cells; leukocytes; lung epithelial cells; exocrine secretory epithelial cells; hormone-secreting cells, keratinocytes; lymphocytes; macrophages; monocytes; renal cells; urethral cells; sensory transducer cells; autonomic neuronal cells; central nervous system neurons; glial cells; skeletal muscle cells; osteocytes; osteoblasts; chondrocytes; smooth muscle cells; microglial cells; stromal cells; or progenitor cells thereof.

44. The method according to any one of claims 40-42, wherein the first plurality of cells comprises hepatocytes and wherein the second plurality of cells comprises neurons.

45. The method according to any one of claims 40-44, wherein stimulating the first plurality of cells involves contacting the first plurality of cells with an agent.

46. The method according to claim 45, wherein the agent is a virus.


SEQUENCE LISTING (5 sequences)

SEQ ID NO: 1 - protein, 448 aa, Artificial sequence
  Synthetic polypeptide
  misc_feature (222)..(222): Xaa can be any amino acid
MGSHHHHHHGMASMTGGQQMGRDLYDDDDKDLATMVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLXVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDKDGDGTITTKELGTVMRSLGQNPTEAELQDMINEVDADGDGTIDFPEFLTMMARKGSYRDTEEEIREAFGVFDKDGNGYISAAELRHVMTNLGEKLTDEEVDEMIREADIDGDGQVNYEEFVQMMTAK

SEQ ID NO: 2 - protein, 15 aa, Artificial sequence
  Synthetic polypeptide
CGGNRWHSIYITRFG

SEQ ID NO: 3 - protein, 15 aa, Artificial sequence
  Synthetic polypeptide
CGGTWYKIAFQRNRK

SEQ ID NO: 4 - protein, 15 aa, Artificial sequence
  Synthetic polypeptide
CGGKAFDITYVRLKF

SEQ ID NO: 5 - protein, 15 aa, Artificial sequence
  Synthetic polypeptide
CGGRKRLQVQLSIRT